(19) United States
(12) Patent Application Publication
(10) Pub. No.: US 2023/0151399 A1
(43) Pub. Date: May 18, 2023

(54) RAW STARCH HYDROLYSIS PROCESS FOR PRODUCING A FERMENTATION PRODUCT

(71) Applicant: Novozymes A/S, Bagsvaerd (DK)

(72) Inventors: Sara Maria Landvik, Vedbaek (DK); Sarah Schultheis Elliott, Raleigh, NC (US); Marie Thryøse Kruse, Vaerloese (DK); Tine Hoff, Vaerloese (DK); Ming Li, Beijing (CN); Tianqi Sun, Aurora (CA)

(73) Assignee: Novozymes A/S, Bagsvaerd (DK)

(21) Appl. No.: 17/798,277

(22) PCT Filed: Feb. 9, 2021
(86) PCT No.: PCT/US2021/017219

§ 371 Date: Aug. 8, 2022

(30) Foreign Application Priority Data
Feb. 10, 2020 (CN) PCT/CN2020/074593
Sep. 4, 2020 (CN) PCT/CN2020/113457

(51) Int. Cl. C12P 19/14; C12N 9/30; C12P 19/02; C12P 7/06

(52) U.S. Cl. CPC C12P 19/14; C12N 9/242; C12P 19/02; C12P 7/06

(57) ABSTRACT

The present invention relates to a raw starch hydrolysis processes for producing a fermentation product.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to a raw starch hydrolysis processes for producing a fermentation product.

### Description of the Related Art

Production of fermentation products, such as ethanol, from starch-containing material is well-known in the art. One well-known process, often referred to as a “raw starch hydrolysis”-process (RSH process), includes simultaneously saccharifying and fermenting granular starch below the initial gelatinization temperature typically in the presence of at least a glucoamylase.

Despite significant improvement of fermentation product production processes over the past decade a significant amount of residual starch material is not converted into the desired fermentation product, such as ethanol.

Therefore, there is still a desire and need for providing raw starch hydrolysis processes for producing fermentation products, such as ethanol, from raw starch material that can provide a higher fermentation product yield, or other advantages, compared to a conventional raw starch hydrolysis process.

### SUMMARY OF THE INVENTION

The present invention relates to a raw starch hydrolysis processes for producing a fermentation product, such as ethanol, from raw starch material using a fermenting organism.

In a first aspect, the invention relates to a raw starch hydrolysis process for producing a fermentation product from a raw starch material comprising the steps of:

(a) saccharifying a raw starch material at a temperature below the initial gelatinization temperature of said raw starch material using a glucoamylase and an alpha-amylase; and

(b) fermenting with a fermenting organism;

wherein the alpha-amylase has at least 60%, e.g., at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 2 or SEQ ID NO: 3.

In a second aspect, the invention relates to a raw starch hydrolysis process for producing a fermentation product from a raw starch material comprising the steps of:

(a) saccharifying a raw starch material at a temperature below the initial gelatinization temperature of said raw starch material using a glucoamylase and an alpha-amylase; and

(b) fermenting with a fermenting organism;

wherein the alpha-amylase has at least 60%, e.g., at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 5 or SEQ ID NO: 6.

In a third aspect, the invention relates to a raw starch hydrolysis process for producing a fermentation product from a raw starch material comprising the steps of:

(a) saccharifying a raw starch material at a temperature below the initial gelatinization temperature of said raw starch material using a glucoamylase and an alpha-amylase; and

(b) fermenting with a fermenting organism;

wherein the alpha-amylase has at least 60%, e.g., at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 8 or SEQ ID NO: 9.

In a fourth aspect, the invention relates to a raw starch hydrolysis process for producing a fermentation product from a raw starch material comprising the steps of:

(a) saccharifying a raw starch material at a temperature below the initial gelatinization temperature of said raw starch material using a glucoamylase and an alpha-amylase; and

(b) fermenting with a fermenting organism;

wherein the alpha-amylase has at least 60%, e.g., at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 11 or SEQ ID NO: 12.

In a fifth aspect, the invention relates to a raw starch hydrolysis process for producing a fermentation product from a raw starch material comprising the steps of:

(a) saccharifying a raw starch material at a temperature below the initial gelatinization temperature of said raw starch material using a glucoamylase and an alpha-amylase; and

(b) fermenting with a fermenting organism;

wherein the alpha-amylase has at least 60%, e.g., at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 14 or SEQ ID NO: 15.

In a sixth aspect, the invention relates to a raw starch hydrolysis process for producing a fermentation product from a raw starch material comprising the steps of:

(a) saccharifying a raw starch material at a temperature below the initial gelatinization temperature of said raw starch material using a glucoamylase and an alpha-amylase; and

(b) fermenting with a fermenting organism;

wherein the alpha-amylase has at least 60%, e.g., at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 16 or SEQ ID NO: 17.

In a seventh aspect, the invention relates to a raw starch hydrolysis process for producing a fermentation product from a raw starch material comprising the steps of:

(a) saccharifying a raw starch material at a temperature below the initial gelatinization temperature of said raw starch material using a glucoamylase and an alpha-amylase; and

(b) fermenting with a fermenting organism;

wherein the alpha-amylase has at least 60%, e.g., at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 20 or SEQ ID NO: 21.

In an eighth aspect, the invention relates to a raw starch hydrolysis process for producing a fermentation product from a raw starch material comprising the steps of:

(a) saccharifying a raw starch material at a temperature below the initial gelatinization temperature of said raw starch material using a glucoamylase and an alpha-amylase; and

(b) fermenting with a fermenting organism;

wherein the alpha-amylase has at least 60%, e.g., at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 23 or SEQ ID NO: 24.

In a ninth aspect, the invention relates to a raw starch hydrolysis process for producing a fermentation product from a raw starch material comprising the steps of:

(a) saccharifying a raw starch material at a temperature below the initial gelatinization temperature of said raw starch material using a glucoamylase and an alpha-amylase; and

(b) fermenting with a fermenting organism;

wherein the alpha-amylase has at least 60%, e.g., at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 26 or SEQ ID NO: 27.

In a tenth aspect, the invention relates to a raw starch hydrolysis process for producing a fermentation product from a raw starch material comprising the steps of:

(a) saccharifying a raw starch material at a temperature below the initial gelatinization temperature of said raw starch material using a glucoamylase and an alpha-amylase; and

(b) fermenting with a fermenting organism;

wherein the alpha-amylase has at least 60%, e.g., at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 29 or SEQ ID NO: 30.

In an eleventh aspect, the invention relates to a raw starch hydrolysis process for producing a fermentation product from a raw starch material comprising the steps of:

(a) saccharifying a raw starch material at a temperature below the initial gelatinization temperature of said raw starch material using a glucoamylase and an alpha-amylase; and

(b) fermenting with a fermenting organism;

wherein the alpha-amylase has at least 60%, e.g., at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 32 or SEQ ID NO: 33.

In a twelfth aspect, the invention relates to a raw starch hydrolysis process for producing a fermentation product from a raw starch material comprising the steps of:

(a) saccharifying a raw starch material at a temperature below the initial gelatinization temperature of said raw starch material using a glucoamylase and an alpha-amylase; and

(b) fermenting with a fermenting organism;

wherein the alpha-amylase has at least 60%, e.g., at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 35 or SEQ ID NO: 36.

In a thirteenth aspect, the invention relates to a raw starch hydrolysis process for producing a fermentation product from a raw starch material comprising the steps of:

(a) saccharifying a raw starch material at a temperature below the initial gelatinization temperature of said raw starch material using a glucoamylase and an alpha-amylase; and

(b) fermenting with a fermenting organism;

wherein the alpha-amylase has at least 60%, e.g., at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 38 or SEQ ID NO: 39.

In an embodiment of each of the first, second, third, fourth, fifth, sixth, seventh, eighth, ninth, tenth, eleventh, twelfth and thirteenth aspects, steps (a) and (b) are carried out simultaneously.

In an embodiment of each of the first, second, third, fourth, fifth, sixth, seventh, eighth, ninth, tenth, eleventh, twelfth and thirteenth aspects, the fermenting organism expresses the alpha-amylase in situ during fermentation.

In an embodiment of each of the first, second, third, fourth, fifth, sixth, seventh, eighth, ninth, tenth, eleventh, twelfth and thirteenth aspects, the fermenting organism expresses the alpha-amylase and the glucoamylase in situ during fermentation.

In an embodiment of each of the first, second, third, fourth, fifth, sixth, seventh, eighth, ninth, tenth, eleventh, twelfth and thirteenth aspects, the fermenting organism is a yeast cell, particularly, a Saccharomyces cell, such as Saccharomyces cerevisiae.

In an embodiment of each of the first, second, third, fourth, fifth, sixth, seventh, eighth, ninth, tenth, eleventh, twelfth and thirteenth aspects, the fermentation product is an alcohol, such as ethanol, preferably fuel ethanol.

### BRIEF DESCRIPTION OF THE FIGURES

FIG. 1 shows percent volume per volume ethanol after 72 h of fermentation in raw-starch slurry with the alpha-amylases of SEQ ID NO: 6, SEQ ID NO: 9, SEQ ID NO: 12, SEQ ID NO: 15, SEQ and SEQ ID NO: 18.

FIG. 2 shows percent volume per volume ethanol after 88 h of fermentation in raw-starch slurry with the alpha-amylases of SEQ ID NO: 9, SEQ ID NO: 12, SEQ ID NO: 18 and SEQ ID NO: 21.

FIG. 3 shows the ethanol kinetics over an 88 h fermentation in raw-starch slurry with the alpha-amylases of SEQ ID NO: 6, SEQ ID NO: 9, SEQ ID NO: 12, SEQ ID NO: 15 and SEQ ID NO: 21.

FIG. 4 shows the percent residual starch after an 88 h fermentation in raw-starch slurry with the alpha-amylases of SEQ ID NO: 6, SEQ ID NO: 9, SEQ ID NO: 12, SEQ ID NO: 15 and SEQ ID NO: 21.

FIG. 5 the percent volume per volume ethanol after 72 h of fermentation in raw-starch slurry for the alpha-amylases of SEQ ID NO: 15, SEQ ID NO: 24, SEQ ID NO: 27, SEQ ID NO: 30 and SEQ ID NO: 33.

FIG. 6 shows the residual starch after 72 h of fermentation in raw-starch slurry for the alpha-amylases of SEQ ID NO: 15, SEQ ID NO: 24, SEQ ID NO: 27, SEQ ID NO: 30 and SEQ ID NO: 33.

FIG. 7 shows the ethanol after 88 h of fermentation in raw-starch slurry for the alpha-amylases of SEQ ID NO: 30, SEQ ID NO: 36, SEQ ID NO: 39 and SEQ ID NO: 40.

FIG. 8 shows the residual starch after 88 h of fermentation in raw-starch slurry for the alpha-amylases of SEQ ID NO: 30, SEQ ID NO: 36, SEQ ID NO: 39 and SEQ ID NO: 40.

### DEFINITIONS

In accordance with this detailed description, the following definitions apply. Note that the singular forms “a,” “an,” and “the” include plural references unless the context clearly dictates otherwise.

Reference to “about” a value or parameter herein includes aspects that are directed to that value or parameter per se. For example, description referring to “about X” includes the aspect “X”.

Unless defined otherwise or clearly indicated by context, all technical and scientific terms used herein have the same meaning as commonly understood by one of ordinary skill in the art to which this invention belongs.

Alpha-amylase: The term “alpha-amylase” means an 1,4-alpha-D-glucan glucanohydrolase, EC. 3.2.1.1, which catalyze hydrolysis of starch and other linear and branched 1,4-glucosidic oligo- and polysaccharides.

For purposes of the present invention, beta-glucosidase activity is determined using p-nitrophenyl-beta-D-glucopyranoside as substrate according to the procedure of Venturi et al., 2002, Extracellular beta-D-glucosidase from Chaetomium thermophilum var. coprophilum: production, purification and some biochemical properties, J. Basic Microbiol. 42: 55-66. One unit of beta-glucosidase is defined as 1.0 μmole of p-nitrophenolate anion produced per minute at 25° C., pH 4.8 from 1 mM p-nitrophenyl-beta-D-glucopyranoside as substrate in 50 mM sodium citrate containing 0.01% TWEEN® 20 (polyoxyethylene sorbitan monolaurate).

Fermentable medium: The term “fermentable medium” or “fermentation medium” refers to a medium comprising one or more (e.g., two, several) sugars, such as glucose, fructose, sucrose, cellobiose, xylose, xylulose, arabinose, mannose, galactose, and/or soluble oligosaccharides, wherein the medium is capable, in part, of being converted (fermented) by a host cell into a desired product, such as ethanol. The term fermentation medium is understood herein to refer to a medium before the fermenting organism is added, such as, a medium resulting from a saccharification process, as well as a medium used in a simultaneous saccharification and fermentation process (SSF).

Glucoamylase: The term “glucoamylase” (1,4-alpha-D-glucan glucohydrolase, EC 3.2.1.3) is defined as an enzyme that catalyzes the release of D-glucose from the non-reducing ends of starch or related oligo- and polysaccharide molecules.

Raw Starch Hydrolysis (RSH): The term “raw starch hydrolysis” means the fermentation of starch ethanol from primary starch based grains which has not been subjected to temperatures above 57° C. for more than 10 minutes.

Sequence identity: The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment).

### DETAILED DESCRIPTION OF THE INVENTION

The present invention relates to a raw starch hydrolysis process for producing a fermentation product, such as ethanol, from a raw starch material using a fermenting organism. The fermentation product, such as ethanol, is produced without liquefying the aqueous slurry containing the raw-starch material and water. The process of the invention includes saccharifying (e.g., milled) raw starch material, e.g., granular starch, below the initial gelatinization temperature, preferably in the presence of an alpha-amylase and glucoamylase to produce sugars that can be fermented into the fermentation product by a suitable fermenting organism. The desired fermentation product, e.g., ethanol, is produced from un-gelatinized (i.e., uncooked), preferably milled, cereal grains, such as corn.

Accordingly, in one aspect the invention relates to a raw starch hydrolysis process for producing a fermentation product from a raw starch material comprising simultaneously saccharifying and fermenting raw starch material using a glucoamylase and a fermenting organism at a temperature below the initial gelatinization temperature of said raw starch material in the presence of a presently disclosed alpha-amylase.

Saccharification and fermentation may also be separate. Thus, in a first aspect, the invention relates to a raw starch hydrolysis process for producing a fermentation product, comprising the steps of:

(a) saccharifying a raw starch material at a temperature below the initial gelatinization temperature of said raw starch material using a glucoamylase and an alpha-amylase; and

(b) fermenting with a fermenting organism;

wherein the alpha-amylase has at least 60%, e.g., at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 2 or SEQ ID NO: 3.

In the second aspect, the invention relates to a raw starch hydrolysis process for producing a fermentation product from a raw starch material comprising the steps of:

(a) saccharifying a raw starch material at a temperature below the initial gelatinization temperature of said raw starch material using a glucoamylase and an alpha-amylase; and

(b) fermenting with a fermenting organism;

wherein the alpha-amylase has at least 60%, e.g., at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 5 or SEQ ID NO: 6.

In a third aspect, the invention relates to a raw starch hydrolysis process for producing a fermentation product from a raw starch material comprising the steps of:

(a) saccharifying a raw starch material at a temperature below the initial gelatinization temperature of said raw starch material using a glucoamylase and an alpha-amylase; and

(b) fermenting with a fermenting organism;

wherein the alpha-amylase has at least 60%, e.g., at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 8 or SEQ ID NO: 9.

In a fourth aspect, the invention relates to a raw starch hydrolysis process for producing a fermentation product from a raw starch material comprising the steps of:

(a) saccharifying a raw starch material at a temperature below the initial gelatinization temperature of said raw starch material using a glucoamylase and an alpha-amylase; and

(b) fermenting with a fermenting organism;

wherein the alpha-amylase has at least 60%, e.g., at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 11 or SEQ ID NO: 12.

In a fifth aspect, the invention relates to a raw starch hydrolysis process for producing a fermentation product from a raw starch material comprising the steps of:

(a) saccharifying a raw starch material at a temperature below the initial gelatinization temperature of said raw starch material using a glucoamylase and an alpha-amylase; and

(b) fermenting with a fermenting organism;

wherein the alpha-amylase has at least 60%, e.g., at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 14 or SEQ ID NO: 15.

In a sixth aspect, the invention relates to a raw starch hydrolysis process for producing a fermentation product from a raw starch material comprising the steps of:

(a) saccharifying a raw starch material at a temperature below the initial gelatinization temperature of said raw starch material using a glucoamylase and an alpha-amylase; and

(b) fermenting with a fermenting organism;

wherein the alpha-amylase has at least 60%, e.g., at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 16 or SEQ ID NO: 17.

In a seventh aspect, the invention relates to a raw starch hydrolysis process for producing a fermentation product from a raw starch material comprising the steps of:

(a) saccharifying a raw starch material at a temperature below the initial gelatinization temperature of said raw starch material using a glucoamylase and an alpha-amylase; and

(b) fermenting with a fermenting organism;

wherein the alpha-amylase has at least 60%, e.g., at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 20 or SEQ ID NO: 21.

In an eighth aspect, the invention relates to a raw starch hydrolysis process for producing a fermentation product from a raw starch material comprising the steps of:

(a) saccharifying a raw starch material at a temperature below the initial gelatinization temperature of said raw starch material using a glucoamylase and an alpha-amylase; and

(b) fermenting with a fermenting organism;

wherein the alpha-amylase has at least 60%, e.g., at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 23 or SEQ ID NO: 24.

In a ninth aspect, the invention relates to a raw starch hydrolysis process for producing a fermentation product from a raw starch material comprising the steps of:

(a) saccharifying a raw starch material at a temperature below the initial gelatinization temperature of said raw starch material using a glucoamylase and an alpha-amylase; and

(b) fermenting with a fermenting organism;

wherein the alpha-amylase has at least 60%, e.g., at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 26 or SEQ ID NO: 27.

In a tenth aspect, the invention relates to a raw starch hydrolysis process for producing a fermentation product from a raw starch material comprising the steps of:

(a) saccharifying a raw starch material at a temperature below the initial gelatinization temperature of said raw starch material using a glucoamylase and an alpha-amylase; and

(b) fermenting with a fermenting organism;

wherein the alpha-amylase has at least 60%, e.g., at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 29 or SEQ ID NO: 30.

In an eleventh aspect, the invention relates to a raw starch hydrolysis process for producing a fermentation product from a raw starch material comprising the steps of:

(a) saccharifying a raw starch material at a temperature below the initial gelatinization temperature of said raw starch material using a glucoamylase and an alpha-amylase; and

(b) fermenting with a fermenting organism;

wherein the alpha-amylase has at least 60%, e.g., at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 32 or SEQ ID NO: 33.

In a twelfth aspect, the invention relates to a raw starch hydrolysis process for producing a fermentation product from a raw starch material comprising the steps of:

(a) saccharifying a raw starch material at a temperature below the initial gelatinization temperature of said raw starch material using a glucoamylase and an alpha-amylase; and

(b) fermenting with a fermenting organism;

wherein the alpha-amylase has at least 60%, e.g., at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 35 or SEQ ID NO: 36.

In a thirteenth aspect, the invention relates to a raw starch hydrolysis process for producing a fermentation product from a raw starch material comprising the steps of:

(a) saccharifying a raw starch material at a temperature below the initial gelatinization temperature of said raw starch material using a glucoamylase and an alpha-amylase; and

(b) fermenting with a fermenting organism;

wherein the alpha-amylase has at least 60%, e.g., at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 38 or SEQ ID NO: 39.

In an embodiment of the first, second, third, fourth, fifth, sixth, seventh, eighth, ninth, tenth, eleventh, twelfth and thirteenth aspects, steps (a) and (b) are carried out simultaneously. In an embodiment of the first, second, third, fourth, fifth, sixth and seventh aspects, the fermenting organism expresses the alpha-amylase in situ during fermentation. In an embodiment of the first, second, third, fourth, fifth, sixth, seventh, eighth, ninth, tenth, eleventh, twelfth and thirteenth aspects, the fermenting organism expresses the alpha-amylase and the glucoamylase in situ during fermentation. In an embodiment of the first, second, third, fourth, fifth, sixth, seventh, eighth, ninth, tenth, eleventh, twelfth and thirteenth aspects, the fermenting organism is a yeast cell, particularly, a Saccharomyces cell, such as Saccharomyces cerevisiae. In an embodiment of the first, second, third, fourth, fifth, sixth, seventh, eighth, ninth, tenth, eleventh, twelfth and thirteenth aspects, the fermentation product is an alcohol, such as ethanol, preferably fuel ethanol.

The fermentation product, e.g., ethanol, may optionally be recovered after fermentation, e.g., by distillation. Typically, amylase(s), such as glucoamylase(s) and/or other carbohydrate-source generating enzymes, and/or alpha-amylase(s), is(are) present during fermentation. Glucoamylases and other carbohydrate-source generating enzymes include raw starch hydrolyzing glucoamylases. Exemplary alpha-amylase(s) include acid fungal alpha-amylases. Examples of fermenting organisms include yeast, e.g., a strain of Saccharomyces cerevisiae. The term “initial gelatinization temperature” means the lowest temperature at which starch gelatinization commences. In general, starch heated in water begins to gelatinize between about 50° C. and 75° C.; the exact temperature of gelatinization depends on the specific starch and can readily be determined by the skilled artisan. Thus, the initial gelatinization temperature may vary according to the plant species, to the particular variety of the plant species as well as with the growth conditions. In the context of this invention the initial gelatinization temperature of a given raw starch material may be determined as the temperature at which birefringence is lost in 5% of the starch granules using the method described by Gorinstein and Lii, 1992, Starch/Starke 44(12): 461-466. Before initiating the process, a slurry of raw starch material, such as granular starch, having 10-55 w/w % dry solids (DS), preferably 25-45 w/w % dry solids, more preferably 30-40 w/w % dry solids of raw starch material may be prepared. The slurry may include water and/or process waters, such as stillage (backset), scrubber water, evaporator condensate or distillate, side-stripper water from distillation, or process water from other fermentation product plants. Because the process of the invention is carried out below the initial gelatinization temperature, and thus no significant viscosity increase takes place, high levels of stillage may be used if desired. In an embodiment the aqueous slurry contains from about 1 to about 70 vol. %, preferably 15-60 vol. %, especially from about 30 to 50 vol. % water and/or process waters, such as stillage (backset), scrubber water, evaporator condensate or distillate, side-stripper water from distillation, or process water from other fermentation product plants, or combinations thereof, or the like. The raw starch material may be prepared by reducing the particle size, preferably by dry or wet milling, to 0.05 to 3.0 mm, preferably 0.1-0.5 mm. After being subjected to a process of the invention at least 85%, at least 86%, at least 87%, at least 88%, at least 89%, at least 90%, at least 91%, at least 92%, at least 93%, at least 94%, at least 95%, at least 96%, at least 97%, at least 98%, or preferably at least 99% of the dry solids in the raw starch material is converted into a soluble starch hydrolyzate. A process in this aspect of the invention is conducted at a temperature below the initial gelatinization temperature, which means that the temperature typically lies in the range between 30-75° C., preferably between 45-60° C. In a preferred embodiment the process carried at a temperature from 25° C. to 40° C., such as from 28° C. to 35° C., such as from 30° C. to 34° C., preferably around 32° C. In an embodiment the process is carried out so that the sugar level, such as glucose level, is kept at a low level, such as below 6 w/w %, such as below about 3 w/w %, such as below about 2 w/w %, such as below about 1 w/w %., such as below about 0.5 w/w %, or below 0.25 w/w %, such as below about 0.1 w/w %. Such low levels of sugar can be accomplished by simply employing adjusted quantities of enzyme and fermenting organism. A skilled person in the art can easily determine which doses/quantities of enzyme and fermenting organism to use. The employed quantities of enzyme and fermenting organism may also be selected to maintain low concentrations of maltose in the fermentation broth. For instance, the maltose level may be kept below about 0.5 w/w %, such as below about 0.2 w/w %. The process of the invention may be carried out at a pH from about 3 and 7, preferably from pH 3.5 to 6, or more preferably from pH 4 to 5. In an embodiment fermentation is ongoing for 6 to 120 hours, in particular 24 to 96 hours.

The alpha-amylase present or added during fermentation or simultaneous saccharification and fermentation may be obtained from microorganisms of the genus Penicillium, e.g., a polypeptide obtained from Penicillium oxalicum, Penicillium sclerotiorum, or Penicillium wotroi. In an embodiment, the alpha-amylase present or added during fermentation or simultaneous saccharification and fermentation is a Penicillium oxalicum polypeptide, for instance, the Penicillium oxalicum polypeptide having alpha-amylase activity of SEQ ID NO: 2 or SEQ ID NO: 3, or a polypeptide having at least 92%, at least 93%, at least 94%, at least 95%, at least 96%, at least 97%, at least 98%, at least 99% amino acid sequence identity to SEQ ID NO: 2 or SEQ ID NO: 3. In an embodiment, the alpha-amylase present or added during fermentation or simultaneous saccharification and fermentation is a Penicillium oxalicum polypeptide, for instance, the Penicillium oxalicum polypeptide having alpha-amylase activity of SEQ ID NO: 2 or SEQ ID NO: 3, or a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to SEQ ID NO: 2 or SEQ ID NO: 3. In an aspect, the alpha-amylase present or added during fermentation or simultaneous saccharification and fermentation is a Penicillium sclerotiorum polypeptide, for instance, the Penicillium sclerotiorum polypeptide having alpha-amylase activity of SEQ ID NO: 5 or SEQ ID NO: 6, or a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to SEQ ID NO: 5 or SEQ ID NO: 6. In an embodiment, the alpha-amylase present or added during fermentation or simultaneous saccharification and fermentation is a Penicillium sclerotiorum polypeptide, for instance, the Penicillium sclerotiorum polypeptide having alpha-amylase activity of SEQ ID NO: 5 or SEQ ID NO: 6, or a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to SEQ ID NO: 5 or SEQ ID NO: 6. In an embodiment, the alpha-amylase present or added during fermentation or simultaneous saccharification and fermentation is a Penicillium wotroi polypeptide, for instance, the Penicillium wotroi polypeptide having alpha-amylase activity of SEQ ID NO: 8 or SEQ ID NO: 9, or a polypeptide having at least 93%, at least 94%, at least 95%, at least 96%, at least 97%, at least 98%, at least 99% amino acid sequence identity to SEQ ID NO: 8 or SEQ ID NO: 9. In an embodiment, the alpha-amylase present or added during fermentation or simultaneous saccharification and fermentation is a Penicillium wotroi polypeptide, for instance, the Penicillium wotroi polypeptide having alpha-amylase activity of SEQ ID NO: 8 or SEQ ID NO: 9, or a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% to SEQ ID NO: 8 or SEQ ID NO: 9.

The alpha-amylase present or added during fermentation or simultaneous saccharification and fermentation may be obtained from the genus Talaromyces, e.g., a polypeptide obtained from Talaromyces helicus. In an embodiment, the alpha-amylase present or added during fermentation or simultaneous saccharification and fermentation is a Talaromyces helicus polypeptide, for instance, the Talaromyces helicus polypeptide having alpha-amylase activity of SEQ ID NO: 11 or SEQ ID NO: 12, or a polypeptide having at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to SEQ ID NO: 11 or SEQ ID NO: 12. In an embodiment, the alpha-amylase present or added during fermentation or simultaneous saccharification and fermentation is a Talaromyces helicus polypeptide, for instance, the Talaromyces helicus polypeptide having alpha-amylase activity of SEQ ID NO: 11 or SEQ ID NO: 12, or a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to SEQ ID NO: 11 or SEQ ID NO: 12.

The alpha-amylase present or added during fermentation or simultaneous saccharification and fermentation may be obtained from the genus Lactobacillus, e.g., a polypeptide obtained from Lactobacillus amylovorus. In an embodiment, the alpha-amylase present or added during fermentation or simultaneous saccharification and fermentation is a Lactobacillus amylovorus polypeptide, for instance, the Lactobacillus amylovorus polypeptide having alpha-amylase activity of SEQ ID NO: 14, SEQ ID NO: 15, SEQ ID NO: 23, SEQ ID NO: 24, SEQ ID NO: 26, SEQ ID NO: 27, SEQ ID NO: 29, SEQ ID NO: 30, SEQ ID NO: 32, or SEQ ID NO: 33, SEQ ID NO: 36, SEQ ID NO: 39, or a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to SEQ ID NO: 14, SEQ ID NO: 15, SEQ ID NO: 23, SEQ ID NO: 24, SEQ ID NO: 26, SEQ ID NO: 27, SEQ ID NO: 29, SEQ ID NO: 30, SEQ ID NO: 32, SEQ ID NO: 33, SEQ ID NO: 36, or SEQ ID NO: 39.

In an embodiment, the alpha-amylase present or added during fermentation or simultaneous saccharification and fermentation is a recombinant polypeptide comprising a Lactobacillus amylovorus catalytic domain having alpha-amylase activity of amino acids 30 to 469 of SEQ ID NO: 14, or amino acids 45 to 467 of SEQ ID NO: 23, SEQ ID NO: 26, SEQ ID NO: 29, SEQ ID NO: 32, SEQ ID NO: 35, and SEQ ID NO: 39, or a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to amino acids 30 to 469 of SEQ ID NO: 14, or amino acids 45 to 467 of SEQ ID NO: 23, SEQ ID NO: 26, SEQ ID NO: 29, SEQ ID NO: 32, SEQ ID NO: 35, and SEQ ID NO: 39, and a His-tag (e.g., C-terminal) comprising at least one, at least two, at least three, at least four, at least five, or at least six C-terminal histidine residues.

In an embodiment, the recombinant polypeptide comprises a C-terminal His-tag consisting of amino acids 470-475 of SEQ ID NO: SEQ ID NO: 14 or amino acids 441 to 446 of SEQ ID NO: 15. In an embodiment, the recombinant polypeptide has a heterologous secretion signal, such as the Bacillus licheniformis secretion signal consisting of an amino acid sequence of amino acids 1-29 of SEQ ID NO: 14 or an amino acid sequence having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to amino acids 1-29 of SEQ ID NO: 14.

The alpha-amylase present or added during fermentation or simultaneous saccharification and fermentation may be obtained from the genus Valsaria, e.g., a polypeptide obtained from Valsaria rubricosa. In an embodiment, the alpha-amylase present or added during fermentation or simultaneous saccharification and fermentation is a Valsaria rubricosa polypeptide, for instance, the Valsaria rubricosa polypeptide having alpha-amylase activity of SEQ ID NO: 16 or SEQ ID NO: 17, or a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to SEQ ID NO: 16 or SEQ ID NO: 17.

According to the invention at least polypeptide having alpha-amylase activity of the present invention is present or added during fermentation or simultaneous saccharification and fermentation, however, preferred embodiments may also include the addition of other enzyme classes during fermentation/SSF. Examples of additional enzymes that can be used include, without limitation, cellulases, glucoamylases, proteases, etc.

In an embodiment, the process of the invention further comprises, prior to the step i), the steps of:

a) reducing the particle size of the raw starch material, preferably by dry milling;

b) forming a slurry comprising the raw starch material and water.

The raw starch starting material, such as whole grains, may be reduced in particle size, e.g., by milling, to open up the structure, to increase surface area, and allowing for further processing. Generally, there are two types of processes: wet and dry milling. Both dry and wet milling are well known in the art of starch processing. According to the present invention dry milling is preferred.

### Saccharification and Fermentation

One or more glucoamylases may be present and/or added during saccharification step ii) and/or fermentation step iii).

When doing sequential saccharification and fermentation, saccharification step ii) may be carried out at conditions well-known in the art. For instance, the saccharification step ii) may last up to from about 24 to about 72 hours. In an embodiment, pre-saccharification is done. Pre-saccharification is typically done for 40-90 minutes at a temperature between 30-65° C., typically about 60° C. Pre-saccharification is in an embodiment followed by saccharification during fermentation in simultaneous saccharification and fermentation (“SSF). Saccharification is typically carried out at temperatures from 20-75° C., preferably from 40-70° C., typically around 60° C., and at a pH between 4 and 5, normally at about pH 4.5.

Simultaneous saccharification and fermentation (“SSF”) is widely used in industrial scale fermentation product production processes, especially ethanol production processes. When doing SSF the saccharification step ii) and the fermentation step iii) are carried out simultaneously. There is no holding stage for the saccharification, meaning that a fermenting organism, such as yeast, and enzyme(s), may be added together. However, it is also contemplated to add the fermenting organism and enzyme(s) separately. SSF is according to the invention typically carried out at a temperature from 25° C. to 40° C., such as from 28° C. to 35° C., such as from 30° C. to 34° C., preferably around about 32° C. In an embodiment fermentation is ongoing for 6 to 120 hours, in particular 24 to 96 hours. In an embodiment the pH is between 3.5-5, in particular between 3.8 and 4.3.

### Fermentation Medium

“Fermentation media” or “fermentation medium” refers to the environment in which fermentation is carried out. The fermentation medium includes the fermentation substrate, that is, the carbohydrate source that is metabolized by the fermenting organism. According to the invention the fermentation medium may comprise nutrients and growth stimulator(s) for the fermenting organism(s). Nutrient and growth stimulators are widely used in the art of fermentation and include nitrogen sources, such as ammonia; urea, vitamins and minerals, or combinations thereof.

### Fermenting Organisms

The term “Fermenting organism” refers to any organism, including bacterial and fungal organisms, especially yeast, suitable for use in a fermentation process and capable of producing the desired fermentation product. Especially suitable fermenting organisms are able to ferment, i.e., convert, sugars, such as glucose or maltose, directly or indirectly into the desired fermentation product, such as ethanol. Examples of fermenting organisms include fungal organisms, such as yeast. Preferred yeast includes strains of Saccharomyces spp., in particular, Saccharomyces cerevisiae.

Suitable concentrations of the viable fermenting organism during fermentation, such as SSF, are well known in the art or can easily be determined by the skilled person in the art. In one embodiment the fermenting organism, such as ethanol fermenting yeast, (e.g., Saccharomyces cerevisiae) is added to the fermentation medium so that the viable fermenting organism, such as yeast, count per mL of fermentation medium is in the range from 10⁵ to 10¹², preferably from 10⁷ to 10¹⁰, especially about 5×10⁷.

Examples of commercially available yeast includes, e.g., RED STAR™ and ETHANOL RED™ yeast (available from Fermentis/Lesaffre, USA), FALI (available from Fleischmann's Yeast, USA), SUPERSTART and THERMOSACC™ fresh yeast (available from Ethanol Technology, WI, USA), BIOFERM AFT and XR (available from NABC—North American Bioproducts Corporation, GA, USA), GERT STRAND (available from Gert Strand AB, Sweden), and FERMIOL (available from DSM Specialties). Other useful yeast strains are available from biological depositories such as the American Type Culture Collection (ATCC) or the Deutsche Sammlung von Mikroorganismen and Zellkulturen GmbH (DSMZ), such as, e.g., BY4741 (e.g., ATCC 201388); Y108-1 (ATCC PTA.10567) and NRRL YB-1952 (ARS Culture Collection). Still other S. cerevisiae strains suitable as host cells DBY746, [Alpha][Eta]22, 5150-2B, GPY55-15Ba, CEN.PK, USM21, TMB3500, TMB3400, VTT-A-63015, VTT-A-85068, VTT-c-79093 and their derivatives as well as Saccharomyces sp. 1400, 424A (LNH-ST), 259A (LNH-ST) and derivatives thereof.

### Raw Starch Materials

Any suitable raw starch starting material may be used according to the present invention. The starting material is generally selected based on the desired fermentation product. Examples of raw starch starting materials, suitable for use in a process of the invention, include whole grains, corn, wheat, barley, rye, milo, sago, cassava, tapioca, sorghum, rice, peas, beans, or sweet potatoes, or mixtures thereof or starches derived there from, or cereals. Contemplated are also waxy and non-waxy types of corn and barley. In a preferred embodiment the raw starch material used for ethanol production according to the invention is corn or wheat.

### Fermentation Products

The term “fermentation product” means a product produced by a process including a fermentation step using a fermenting organism. Fermentation products contemplated according to the invention include alcohols (e.g., ethanol, methanol, butanol; polyols such as glycerol, sorbitol and inositol); organic acids (e.g., citric acid, acetic acid, itaconic acid, lactic acid, succinic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, B₁₂, beta-carotene); and hormones. In a preferred embodiment the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. Preferred beer types comprise ales, stouts, porters, lagers, bitters, malt liquors, happoushu, high-alcohol beer, low-alcohol beer, low-calorie beer or light beer. Preferably processes of the invention are used for producing an alcohol, such as ethanol. The fermentation product, such as ethanol, obtained according to the invention, may be used as fuel, which is typically blended with gasoline. However, in the case of ethanol it may also be used as potable ethanol.

### Recovery

Subsequent to fermentation, or SSF, the fermentation product may be separated from the fermentation medium. The slurry may be distilled to extract the desired fermentation product (e.g., ethanol). Alternatively, the desired fermentation product may be extracted from the fermentation medium by micro or membrane filtration techniques. The fermentation product may also be recovered by stripping or other method well known in the art.

Glucoamylase Present and/or Added During Saccharification and/or Fermentation

According to the invention a glucoamylase may be present and/or added during saccharification and/or fermentation. The glucoamylase present and/or added during saccharification and/or fermentation may be derived from any suitable source, e.g., derived from a microorganism or a plant. Preferred glucoamylases are of fungal or bacterial origin, such as for instance from a stain of Aspergillus, preferably A. niger, A. awamori, or A. oryzae; or a strain of Trichoderma, preferably T. reesei; or a strain of Talaromyces, preferably T. emersonii, or a strain of Trametes, preferably Trametes cingulata, or a strain of Pycnoporus, or a strain of Gloeophyllum, such as a strain of Gloeophyllum sepiarium or Gloeophyllum trabeum or a strain of the Nigrofomes.

In an embodiment the glucoamylase is derived from Trametes, such as a strain of Trametes cingulata, such as the one shown in SEQ ID NO: 18 herein.

In an embodiment the glucoamylase is selected from the group consisting of:

a glucoamylase comprising the polypeptide of SEQ ID NO: 18 herein;

(ii) a glucoamylase comprising an amino acid sequence having at least 60%, at least 70%, e.g., at least 75%, at least 80%, at least 85%, at least 90%, at least 91%, at least 92%, at least 93%, at least 94%, at least 95%, at least 96%, at least 97%, at least 98%, or at least 99% identity to the polypeptide of SEQ ID NO: 7 herein.

Glucoamylases may in an embodiment be added to the saccharification and/or fermentation in an amount of 0.0001-20 AGU/g DS, preferably 0.001-10 AGU/g DS, especially between 0.01-5 AGU/g DS, such as 0.1-2 AGU/g DS.

In an embodiment the glucoamylase is added as a blend further comprising a presently disclosed alpha-amylase.

Commercially available compositions comprising glucoamylase include AMG 200L; AMG 300 L; SAN™ SUPER, SAN™ EXTRA L, SPIRIZYME™ PLUS, SPIRIZYME™ FUEL, SPIRIZYME™ B4U, SPIRIZYME™ ULTRA, SPIRIZYME™ EXCEL, SPIRIZYME ACHIEVE and AMG™ E (from Novozymes A/S); OPTIDEX™ 300, GC480, GC417 (from DuPont-Genencor); AMIGASE™ and AMIGASE™ PLUS (from DSM); G-ZYME™ G900, G-ZYME™ and G990 ZR (from DuPont-Genencor).

### EXAMPLES

### Materials & Methods

Saccharomyces cerevisiae strain MBG5012: Saccharomyces cerevisiae strain MBG5012 (deposited under Accession No. NRRL Y67549 at the Agricultural Research Service Patent Culture Collection (NRRL), Northern Regional Research Center, 1815 University Street, Peoria, Ill., USA).

SEQ ID NO: 6: polypeptide having alpha-amylase activity from Penicillium sclerotiorum.

SEQ ID NO: 9: polypeptide having alpha-amylase activity from Penicillium wotroi.

SEQ ID NO: 12: polypeptide having alpha-amylase activity from Talaromyces helices.

SEQ ID NO: 15: polypeptide having alpha-amylase activity from Lactobacillus amylovorus.

SEQ ID NO: 18: polypeptide having alpha-amylase activity from Valsaria rubricosa.

SEQ ID NO: 21: polypeptide having alpha-amylase activity from Bacillus amyloliquefaciens.

SEQ ID NO: 24: polypeptide having alpha-amylase activity comprising catalytic domain from Lactobacillus amylovorus.

SEQ ID NO: 27: polypeptide having alpha-amylase activity comprising catalytic domain from Lactobacillus amylovorus.

SEQ ID NO: 30: polypeptide having alpha-amylase activity comprising catalytic domain from Lactobacillus amylovorus.

SEQ ID NO: 33: polypeptide having alpha-amylase activity comprising catalytic domain from Lactobacillus amylovorus.

SEQ ID NO: 36: polypeptide having alpha-amylase activity comprising catalytic domain from Lactobacillus amylovorus.

SEQ ID NO: 39: polypeptide having alpha-amylase activity comprising catalytic domain from Lactobacillus amylovorus.

SEQ ID NO: 40: Rhizomucor pusillus alpha-amylase with an Aspergillus niger glucoamylase linker and starch-binding domain (SBD), disclosed SEQ ID NO: 3 in WO 2013/006756 (SEQ ID NO: 32 herein) with the following substitutions: G128D+D143N.

Tc AMG: Trametes cingulata glucoamylase disclosed in SEQ ID NO: 18.

The present invention is described in further detail in the following examples which are offered to illustrate the present invention, but not in any way intended to limit the scope of the invention as claimed. All references cited herein are specifically incorporated by reference for that which is described therein.

### Examples

### Example 1—Evaluation of Effect of Alpha-Amylases on Ethanol Yield During Simultaneous Saccharification and Fermentation (SSF) of Raw Starch Corn Mash

This example describes the evaluation of the alpha-amylases with SEQ ID NO: 6, SEQ ID NO: 9, SEQ ID NO: 12, SEQ ID NO: 15, SEQ ID NO: 18, and SEQ ID NO: 21 (“alpha-amylases”) for improved ethanol during raw starch corn mash SSF. Particularly, the ethanol during fermentation is compared among the alpha-amylases.

### Seed Culture

Cryo-preserved culture of fermenting organism Saccharomyces cerevisiae strain MBG5012 was first grown in liquid YPD media (Yeast extract, 10 g. Peptone, 20 g. Dextrose, 60 g. dissolve in 1 L of distilled water). Cultivation was done aseptically in a sterile 125-ml Erlenmeyer flask filled with 50 ml YPD media and inoculated with 100 μl of cryo-preserved culture. Flask was incubated in a shaking incubator at 32° C. for 16 h with shaking at 150 rpm. The YPD grown seed cultures (40 ml) were centrifuged at 3,500 rpm for 10 min at 22° C., and the resulting cell pellet was washed and resuspended in tap water and glycerol. The resuspended cells were used to inoculate the corn mash at the beginning of simultaneous saccharification and fermentation (SSF).

### Corn Mash

Corn kernels were ground using the Turkish grind setting on a Bunn Coffee Grinder. The % dried solids (DS) of the corn flour was 84.50%. Using the Turkish ground corn and tap water, slurries targeting either 37% DS or 34.5% DS were prepared. Each corn slurry was supplemented with 1000 ppm urea and 3 ppm of antibiotic LACTROL™ and the pH was adjusted to 4.5 prior to use in SSF. Final dried solids level was determined to be 37.09% DS and 34.52% DS, respectively.

### Simultaneous Saccharification and Fermentation (SSF)

All fermentations were carried out in 12 mL round-bottom tubes with caps having a drilled hole. Tubes were filled with 3.8-4.5 g of corn slurry and inoculated with seed culture at 10 million cells per gram mash. A glucoamylase (TcAMG) was added to the tubes at 88 μg enzyme protein per g of dry corn solids. The alpha-amylases were added to the tubes at 32 μg enzyme protein per g of dry corn solids. All tubes contained the same glucoamylase and one of the alpha-amylases was added per tube. Tubes were incubated in an incubator at 32° C. Tubes were vortexed two times per day. Fermentations using the 37.09% DS slurry were run for 72 hours and fermentations using the 34.52% DS slurry were run for 88 hours.

### Ethanol Analysis

After 72 hours or 88 hours of fermentation, 100 μL of 40% v/v H₂SO₄ was added to each sample tube, samples were vortexed, and centrifuged at 3,500 rpm for 10 min at 22° C. The resulting supernatant was filtered through a 0.2 μm syringe filter. Filtered samples were stored at 4° C. prior to and during HPLC analysis. Analysis of ethanol was conducted using an HPLC (Agilent 1100/1200 series) machine equipped with a guard column (Bio-Rad, Micro-Guard Cation H+ Cartridge, 30×4.6 mm) and an analytical column (Bio-Rad, Aminex HPX-87H, 300×7.8 mm) using 5 mM Sulfuric Acid as a mobile phase with a flow rate of 0.8 mL/min. Column temperature was maintained at 65° C., and ethanol was detected using a Refractive Index detector at 55° C.

### Results

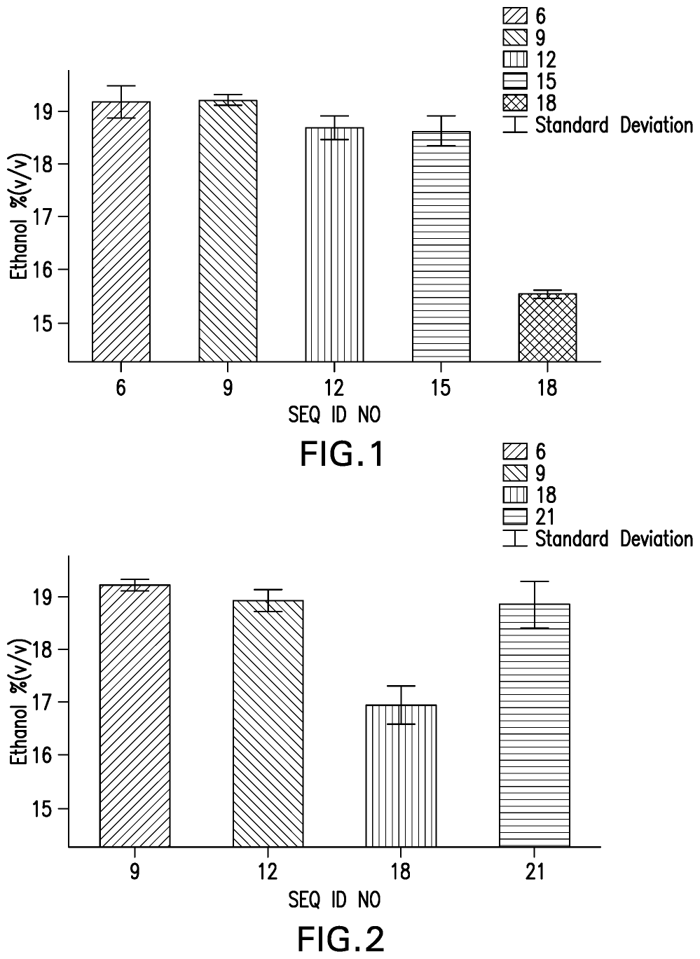

FIG. 1 and FIG. 2 show ethanol titers after 72 h and 88 h of fermentations, respectively, for the alpha-amylases during corn mash fermentation. Results indicate ethanol titer increase for fermentations using SEQ ID NO: 6, SEQ ID NO: 9, SEQ ID NO: 12, 1 SEQ ID NO: 5 and SEQ ID NO: 21 versus SEQ ID NO: 18.

### Example 2—Evaluation of Effect of Alpha-Amylases on Ethanol Kinetics and Residual Starch Following RSH SSF

This example shows ethanol kinetics and the amount of starch remaining following an 88 hour RSH SSF with SEQ ID NO: 6, SEQ ID NO: 9, SEQ ID NO: 12, SEQ ID NO: 15 and SEQ ID NO: 21 (“alpha-amylases”). For this assay, starch is considered insoluble in aqueous solution, so any soluble glucose/maltodextrin is removed via two additional washes using deionized water. Washed insoluble solid was freeze dried and 0.1 g of this dried material was then subjected to the assay (described below) to obtain the starch of the insoluble solid. The total dry solid was determined in order to convert the starch of insoluble solid to the starch of dry solid. Residual starch level, when treated with various α-amylases, can be compared to the ethanol titer to determine the overall effectiveness of alpha-amylases in accessing starch and converting that to ethanol in RSH SSF.

### Seed Culture

Cryo-preserved culture of fermenting organism Saccharomyces cerevisiae strain MBG5012 was grown in liquid YPD media (Yeast extract, 10 g. Peptone, 20 g. Dextrose, 60 g. dissolve in 1 L of distilled water). Cultivation was done aseptically in a sterile 125-ml Erlenmeyer flask filled with 50 ml YPD media and inoculated with 100 μl of cryo-preserved culture. Flask was incubated in a shaking incubator at 32° C. for 16 h with shaking at 150 rpm. The YPD grown seed cultures (40 ml) were centrifuged at 3,500 rpm for 10 min at 22° C., and the resulting cell pellet was washed and resuspended in tap water. The resuspended cells were used to inoculate the corn mash at the beginning of simultaneous saccharification and fermentation

### Corn Mash

Corn flour and pre-blend from a commercial plant was mixed. The corn slurry was supplemented with 200 ppm urea and 3 ppm of antibiotic LACTROL™ and its pH was adjusted to 4.5 prior to use in SSF. Final dried solids level was determined to be 37.2% DS.

### Simultaneous Saccharification and Fermentation (SSF)

All fermentations were carried out in 125 mL jars with caps having a drilled hole. Jars were filled with 70-85 g of corn slurry and inoculated with seed culture at 10 million cells per gram mash. A glucoamylase (TcAMG) was added to the jars at 88 μg enzyme protein per g of dry corn solids. Alpha-amylases were added to the jars at 32 μg enzyme protein per g of dry corn solids. All jars contained the same glucoamylase and one of the alpha-amylases. Jars were incubated in a waterbath at 32° C. Jars were swirled two times per day. Fermentation was run for 88 hours.

### Ethanol, Sugars and Organic Acid Analyses

After 24, 48, 72 and 88 h of fermentation, 4 mL samples were taken from each jar, and 100 uL of 40% (v/v) H₂SO₄ was added to stop the reaction. These mixtures were vortexed, and centrifuged at 3,500 rpm for 10 min at 22° C. The resulting supernatant was filtered through a 0.2 μm syringe filter. Filtered samples were stored at 4° C. prior to and during HPLC analysis. Analysis of ethanol, sugars and organic acids was conducted using an HPLC (Agilent 1100/1200 series) machine equipped with a guard column (Bio-Rad, Micro-Guard Cation H+ Cartridge, 30×4.6 mm) and an analytical column (Bio-Rad, Aminex HPX-87H, 300×7.8 mm) using 5 mM Sulfuric Acid as a mobile phase with a flow rate of 0.8 mL/min. Column temperature was maintained at 65° C., and analytes were detected using a Refractive Index detector at 55° C.

### Total Dry Solid Determination

Total dry solid was determined on the 88 h samples by placing ˜1.5 g of fermentation sample (wet weight) onto a pre-weighed aluminum weigh boat and incubated in a forced air oven maintaining at 105° C. for 16-20 hours. The percent of total dry solid was calculated by dividing the dried weight to the wet weight of the sample.

### The Insoluble Solid Determination

The insoluble solid was determined on the 88 h samples by placing between 7-10 grams of fermentation sample into a pre-weighed 15 mL Genogrind vials. Deionized water was added to each vial to the 15 mL total volume and then centrifuged at 3500 rpm for 10 minutes. The supernatant was decanted and the insoluble solid washed two more times by resuspending in deionized (to 15 mL), followed by centrifugation and decanting. The insoluble pellet was placed in −20° C. freezer for >2 hours and then lyophilized for 24 hours using Labconco FreeZone freeze dryer. The weight of the freeze dried sample was measured and the percent insoluble solid was calculated by dividing the freeze dried weight by the wet mash weight.

### Residual Starch Assay

Residual starch is determined following the Megazyme Total Starch (AA/AMG) Kit (Catalog #K-TSTA) with the following modification. The freeze dried sample (from the insoluble solid determination step) was ground to fine powder using the Geno/Grinder Spex Sample Prep at 1600 rpm for 4 minutes. The starch level in this ground sample (insoluble solid) was determined according to the method described in the “Example A” of the kit. After all enzyme treatments, the glucose level was quantitated following the same HPLC method described in “Ethanol, sugars and organic acid analyses” section. Using the percent of insoluble solids and total dry solid, the starch of insoluble solid was converted to the percent starch of dry solid following the calculation described in the Megazyme kit. The value resulting from the calculation is described as “% residual starch” in result section below.

### Results

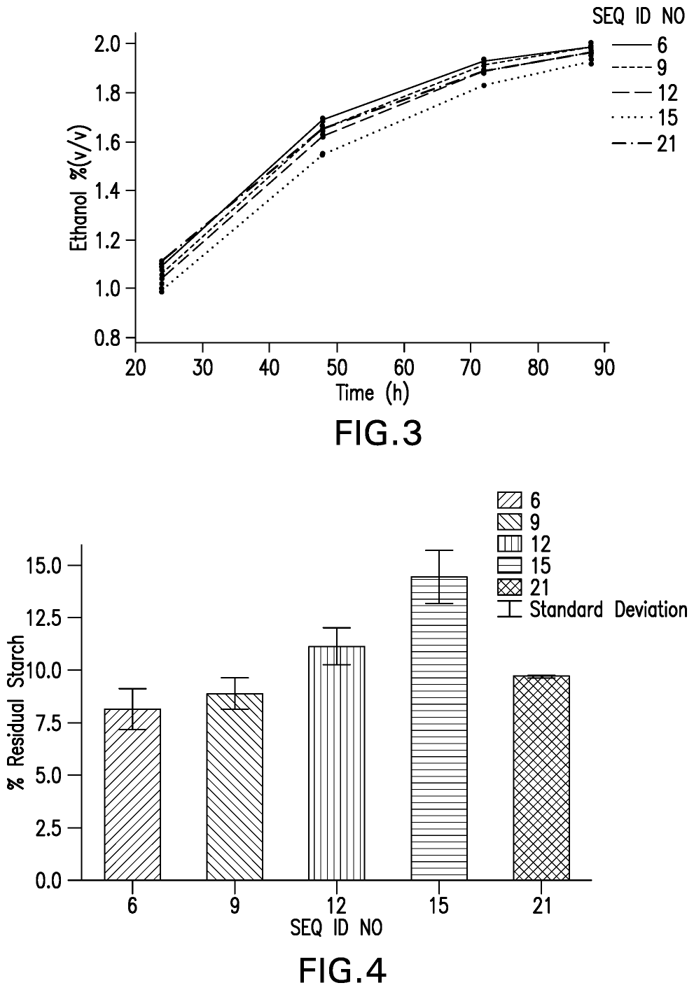

FIG. 3 shows the ethanol kinetics over 88 hours of fermentation by alpha-amylase treatment. FIG. 4 shows the percent residual starch after 88 hours of fermentation by alpha-amylase treatment. In Example 1, fermentation containing SEQ ID NO: 6, SEQ ID NO: 9, SEQ ID NO: 12, SEQ ID NO: 15 or SEQ ID NO: 21 showed improved ethanol versus SEQ ID NO: 18. FIG. 3 shows fermentations with SEQ ID NO: 6, SEQ ID NO: 9, SEQ ID NO: 12 or SEQ ID NO: 21 have faster ethanol kinetics versus SEQ ID NO: 15. FIG. 4 shows lower percent residual starch for fermentations containing SEQ ID NO: 6, SEQ ID NO: 9, SEQ ID NO: 12 or SEQ ID NO: 21 versus SEQ ID NO: 15. Results indicate that alpha-amylases with SEQ ID NO: 6, SEQ ID NO: 9, SEQ ID NO: 12 or SEQ ID NO: 21 improve ethanol kinetics and lower residual starch versus SEQ ID NO: 15.

### Example 3—Evaluation of Effect of Alpha-Amylases on Ethanol Yield Following RSH SSF

This example describes the evaluation of the alpha-amylases of SEQ ID NO: 24, SEQ ID NO: 27, SEQ ID NO: 30 and SEQ ID NO: 33 compared to the alpha-amylase of SEQ ID NO: 15 for improved ethanol yield during raw-starch corn mash SSF.

### Seed Culture:

Cryo-preserved culture of Saccharomyces cerevisiae strain MBG5012 was first grown in liquid YPD media (Yeast extract, 10 g. Peptone, 20 g. Dextrose, 60 g. dissolve in 1 L of distilled water). Cultivation was done aseptically in a sterile 125-ml Erlenmeyer flask filled with 50 ml YPD media and inoculated with 100 μl of cryo-preserved culture. Flask was incubated in a shaking incubator at 32° C. for 16 h with shaking at 150 rpm. The YPD grown seed cultures (40 ml) were centrifuged at 3,500 rpm for 10 min at 22° C., and the resulting cell pellet was washed and resuspended in tap water and glycerol. The resuspended cells were used to inoculate the corn mash at the beginning of simultaneous saccharification and fermentation (SSF).

### Corn Mash:

Corn kernels were ground using the Turkish grind setting on a Bunn Coffee Grinder. The % dried solids (DS) of the corn flour was 84.5%. Using the Turkish ground corn and tap water, a slurry targeting 35.0% DS was prepared. The corn slurry was supplemented with 1000 ppm urea and 3 ppm of antibiotic LACTROL™ and its pH was adjusted to 4.5 prior to use in SSF. Final dried solids (DS) level was determined to be 35.0% DS.

### Simultaneous Saccharification and Fermentation (SSF)

All fermentations were carried out in 12 mL round-bottom tubes with caps having a drilled hole. Tubes were filled with 3.8-4.5 g of corn slurry and inoculated with seed culture at 10 million cells per gram mash. A glucoamylase (TcAMG) was added to the tubes at 88 μg enzyme protein per g of dry corn solids. The alpha-amylases shown in SEQ ID NO: 15, SEQ ID NO: 24, SEQ ID NO: 27, SEQ ID NO: 30 and SEQ ID NO: 33 were added to the tubes at 32 μg enzyme protein per g of dry corn solids. Each tubes contained the same glucoamylase and a different one of the alpha-amylases. Tubes were incubated in an incubator at 32° C. Tubes were vortexed two times per day. Fermentation was run for 72 hours.

### Ethanol Analysis

After 72 hours of fermentation, 100 μL of 40% v/v H₂SO₄ was added to each sample tube, samples were vortexed, and centrifuged at 3,500 rpm for 10 min at 22° C. The resulting supernatant was filtered through a 0.2 μm syringe filter. Filtered samples were stored at 4° C. prior to and during HPLC analysis. Analysis of ethanol was conducted using an HPLC (Agilent 1100/1200 series) machine equipped with a guard column (Bio-Rad, Micro-Guard Cation H+ Cartridge, 30×4.6 mm) and an analytical column (Bio-Rad, Aminex HPX-87H, 300×7.8 mm) using 5 mM Sulfuric Acid as a mobile phase with a flow rate of 0.8 mL/min. Column temperature was maintained at 65° C., and ethanol was detected using a Refractive Index detector at 55° C.

### Results

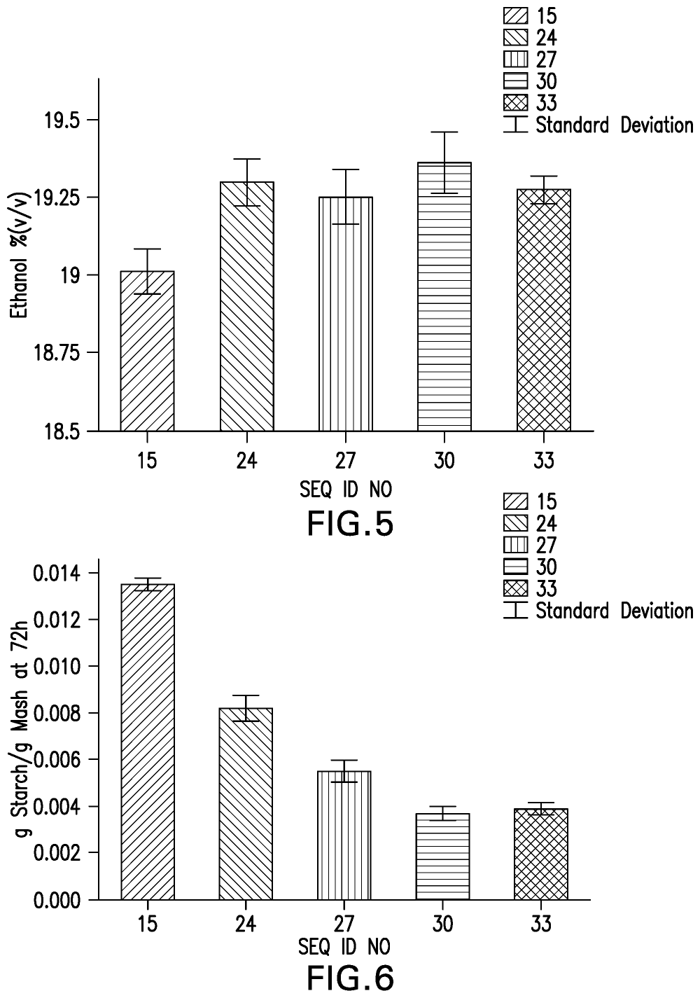

FIG. 5 shows ethanol titers after 72 hours of fermentation by alpha-amylase treatment. Results indicate ethanol titer increase for fermentations containing alpha-amylases with SEQ ID NO: 24, SEQ ID NO: 27, SEQ ID NO: 30 or SEQ ID NO: 33 compared to the fermentation containing the alpha-amylase of SEQ ID NO: 15.

### Example 4—the Effects of Alpha-Amylases on the Residual Starch after RSH SSF

This example provides an estimate of the amount of starch remaining (residual starch) following 72-hour RSH SSF with the alpha-amylases of SEQ ID NO: 24, SEQ ID NO: 27, SEQ ID NO: 30 and SEQ ID NO: 33 compared to the alpha-amylase of SEQ ID NO: 15.

For this assay, starch is considered insoluble in aqueous solution, so any soluble glucose/maltodextrin was removed via two washes using deionized water. The insoluble solid from the fermentation described in Example 3 was pelleted by centrifugation at 3500 rpm for 10 minutes at 22° C. After removing supernatant, the pellet was washed with deionized water two times, centrifuged and supernatant was removed. The insoluble solid was resuspended in 5 mL deionized water and 0.05 mL of Novozymes' Novozym BPX10.5C enzyme product. The mixture was vortexed and incubated for 18 hours at 50° C. Samples were centrifuged at 3,500 rpm for 10 min at 22° C. The resulting supernatant was filtered through a 0.2 μm syringe filter. Filtered samples were stored at 4° C. prior to and during HPLC analysis. Analysis of glucose was conducted using the same HPLC analysis as described in Example 3 above (under the Ethanol Analysis section) but glucose data was used instead of ethanol. The starch was calculated based on resulting HPLC glucose value and using the 0.9 gram of starch/g glucose conversion factor. The starch value was then divided by the grams of corn mash remaining at the end of fermentation, resulting in a residual g starch/g 72 h mash value. The residual starch level, when treated with various alpha-amylases, can be compared amongst enzyme treatments to determine the overall effectiveness of alpha-amylases in accessing starch.

### Results

FIG. 6 shows residual starch (g starch/g mash at 72 h) after 72 hours of fermentation by alpha-amylase treatment. Results indicate lowered residual starch for fermentations with alpha-amylases of SEQ ID NO: 24, SEQ ID NO: 27, SEQ ID NO: 30 or SEQ ID NO: 33 compared to the alpha-amylase of SEQ ID NO: 15. Results indicate that the enzymes can significantly lower residual starch in a corn mash fermentation.

### Example 5—Evaluation of Alpha-Amylases Impact on Ethanol Following RSH SSF

This example shows ethanol following 88 hour RSH SSF with SEQ ID NO: 30, SEQ ID NO: 36, SEQ ID NO: 39 and SEQ ID NO: 40 (“alpha-amylases”).

### Seed Culture:

Cryo-preserved culture of Saccharomyces cerevisiae strain MBG5012 was first grown in liquid YPD media (Yeast extract, 10 g. Peptone, 20 g. Dextrose, 60 g. dissolve in 1 L of distilled water). Cultivation was done aseptically in a sterile 125-ml Erlenmeyer flask filled with 50 ml YPD media and inoculated with 100 μl of cryo-preserved culture. Flask was incubated in a shaking incubator at 32° C. for 16 h with shaking at 150 rpm. The YPD grown seed cultures (40 ml) were centrifuged at 3,500 rpm for 10 min at 22° C., and the resulting cell pellet was washed and resuspended in tap water and glycerol. The resuspended cells were used to inoculate the corn mash at the beginning of simultaneous saccharification and fermentation (SSF).

### Corn Mash

Corn flour and pre-blend from a commercial plant was mixed. The corn slurry was supplemented with 200 ppm urea and 3 ppm of antibiotic LACTROL™ and its pH was adjusted to 4.5 prior to use in SSF. Final dried solids level was determined to be 36.5% DS.

### Simultaneous Saccharification and Fermentation (SSF)

All fermentations were carried out in 125 mL jars with caps having a drilled hole. Jars were filled with 70-85 g of corn slurry and inoculated with seed culture at 10 million cells per gram mash. A glucoamylase (TcAMG) was added to the jars at 88 μg enzyme protein per g of dry corn solids. Alpha-amylases were added to the jars at 32 μg enzyme protein per g of dry corn solids. All jars contained the same glucoamylase and one of the alpha-amylases. Jars were incubated in a waterbath at 32° C. Jars were swirled two times per day. Fermentation was run for 88 hours.

### Ethanol, Sugars and Organic Acid Analyses

After 88 h of fermentation, 4 mL samples were taken from each jar, and 100 uL of 40% (v/v) H₂SO₄ was added to stop the reaction. These mixtures were vortexed, and centrifuged at 3,500 rpm for 10 min at 22° C. The resulting supernatant was filtered through a 0.2 μm syringe filter. Filtered samples were stored at 4° C. prior to and during HPLC analysis. Analysis of ethanol, sugars and organic acids was conducted using an HPLC (Agilent 1100/1200 series) machine equipped with a guard column (Bio-Rad, Micro-Guard Cation H+ Cartridge, 30×4.6 mm) and an analytical column (Bio-Rad, Aminex HPX-87H, 300×7.8 mm) using 5 mM Sulfuric Acid as a mobile phase with a flow rate of 0.8 mL/min. Column temperature was maintained at 65° C., and analytes were detected using a Refractive Index detector at 55° C.

### Results

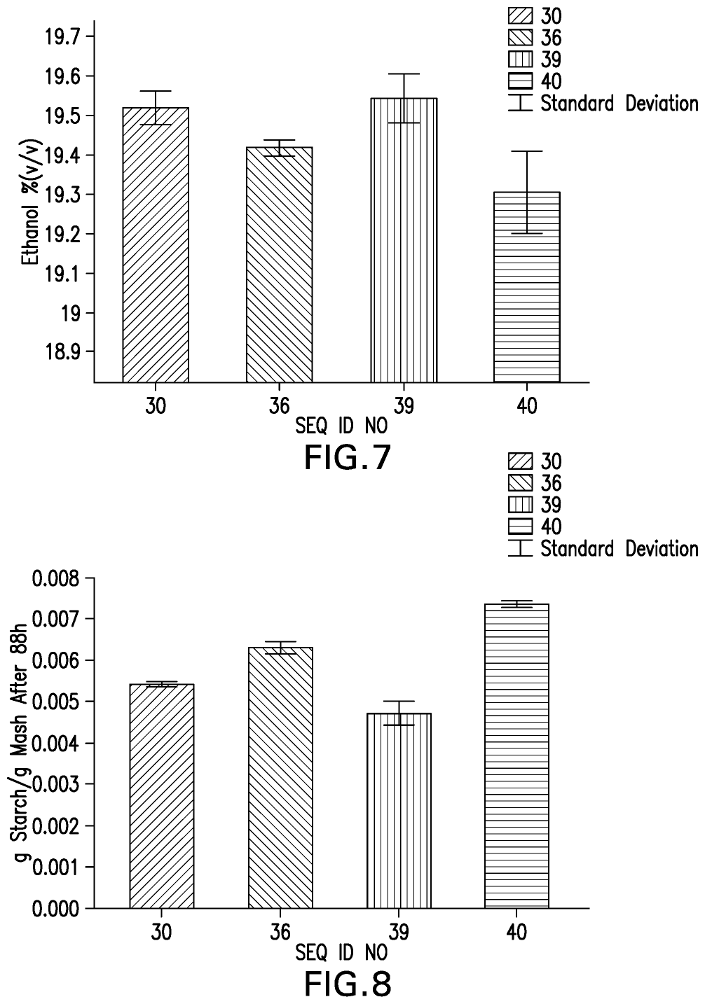

FIG. 7 shows ethanol after 88 hours of fermentation by alpha-amylase treatment. Results show fermentations containing alpha-amylases with SEQ ID NO: 36, SEQ ID NO: 30, or SEQ ID NO: 39 have improved ethanol versus a fermentation with SEQ ID NO: 40.

### Example 6—Evaluation of Impact of Alpha-Amylases on Residual Starch Following RSH SSF

This example provides an estimate of the amount of starch remaining (residual starch) following 88 hour RSH SSF with the alpha-amylases of SEQ ID NO: 30, SEQ ID NO: 36, SEQ ID NO: 39 and SEQ ID NO: 40 (“alpha-amylases”).

For this assay, starch is considered insoluble in aqueous solution, so any soluble glucose/maltodextrin was removed via two washes using deionized water. The insoluble solid from the fermentation described in Example 5 was pelleted by centrifugation at 3500 rpm for 10 minutes at 22° C. After removing supernatant, the pellet was washed with deionized water two times, centrifuged and supernatant was removed. The insoluble solid was resuspended in 3.5 mL deionized water and 0.05 mL of Novozymes' Novozym BPX10.5C enzyme product. The mixture was vortexed and incubated for 24 hours at 50° C. Samples were centrifuged at 3,500 rpm for 10 min at 22° C. The resulting supernatant was filtered through a 0.2 μm syringe filter. Filtered samples were stored at 4° C. prior to and during HPLC analysis. Analysis of glucose was conducted using the same HPLC analysis as described in Example 5 above (under the Ethanol, sugars and organic acid analyses section) but glucose data was used instead of ethanol. The starch was calculated based on resulting HPLC glucose value and using the 0.9 gram of starch/g glucose conversion factor. The starch value was then divided by the grams of corn mash in the sample, resulting in a residual g starch/g 88 h mash value. The residual starch level, when treated with various alpha-amylases, can be compared amongst enzyme treatments to determine the overall effectiveness of alpha-amylases in accessing starch.

### Results

FIG. 8 shows residual starch (g starch/g mash at 88 h) after 88 hours of fermentation by alpha-amylase treatment. Results show fermentations containing SEQ ID NO:30, SEQ ID NO: 36 or SEQ ID NO: 39 result in lower residual starch than fermentations containing SEQ ID NO: 40.

1. A raw starch hydrolysis process for producing a fermentation product from a raw starch material, comprising the steps of:
(a) saccharifying a raw starch material at a temperature below the initial gelatinization temperature of said raw starch material using a glucoamylase and an alpha-amylase; and
(b) fermenting with a fermenting organism;
wherein the alpha-amylase has at least 60%, at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 2 or SEQ ID NO: 3.

2. A raw starch hydrolysis process for producing a fermentation product from a raw starch material comprising the steps of:
(a) saccharifying a raw starch material at a temperature below the initial gelatinization temperature of said raw starch material using a glucoamylase and an alpha-amylase; and
(b) fermenting with a fermenting organism;
wherein the alpha-amylase has at least 60%, at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 5 or SEQ ID NO: 6.

3. A raw starch hydrolysis process for producing a fermentation product from a raw starch material comprising the steps of:
(a) saccharifying a raw starch material at a temperature below the initial gelatinization temperature of said raw starch material using a glucoamylase and an alpha-amylase; and
(b) fermenting with a fermenting organism;
wherein the alpha-amylase has at least 60%, at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 8 or SEQ ID NO: 9.

4. A raw starch hydrolysis process for producing a fermentation product from a raw starch material comprising the steps of:
(a) saccharifying a raw starch material at a temperature below the initial gelatinization temperature of said raw starch material using a glucoamylase and an alpha-amylase; and
(b) fermenting with a fermenting organism;
wherein the alpha-amylase has at least 60%, at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 11 or SEQ ID NO: 12.

5. A raw starch hydrolysis process for producing a fermentation product from a raw starch material comprising the steps of:
(a) saccharifying a raw starch material at a temperature below the initial gelatinization temperature of said raw starch material using a glucoamylase and an alpha-amylase; and
(b) fermenting with a fermenting organism;
wherein the alpha-amylase has at least 60%, at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 14 or SEQ ID NO: 15.

6. A raw starch hydrolysis process for producing a fermentation product from a raw starch material comprising the steps of:
(a) saccharifying a raw starch material at a temperature below the initial gelatinization temperature of said raw starch material using a glucoamylase and an alpha-amylase; and
(b) fermenting with a fermenting organism;
wherein the alpha-amylase has at least 60%, at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 16 or SEQ ID NO: 17.

7. A raw starch hydrolysis process for producing a fermentation product from a raw starch material comprising the steps of:
(a) saccharifying a raw starch material at a temperature below the initial gelatinization temperature of said raw starch material using a glucoamylase and an alpha-amylase; and
(b) fermenting with a fermenting organism;
wherein the alpha-amylase has at least 60%, at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 20 or SEQ ID NO: 21.

8. A raw starch hydrolysis process for producing a fermentation product from a raw starch material comprising the steps of:
(a) saccharifying a raw starch material at a temperature below the initial gelatinization temperature of said raw starch material using a glucoamylase and an alpha-amylase; and
(b) fermenting with a fermenting organism;
wherein the alpha-amylase has at least 60%, at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 23 or SEQ ID NO: 24.

9. A raw starch hydrolysis process for producing a fermentation product from a raw starch material comprising the steps of:
(a) saccharifying a raw starch material at a temperature below the initial gelatinization temperature of said raw starch material using a glucoamylase and an alpha-amylase; and
(b) fermenting with a fermenting organism;
wherein the alpha-amylase has at least 60%, at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 26 or SEQ ID NO: 27.

10. A raw starch hydrolysis process for producing a fermentation product from a raw starch material comprising the steps of:
(a) saccharifying a raw starch material at a temperature below the initial gelatinization temperature of said raw starch material using a glucoamylase and an alpha-amylase; and
(b) fermenting with a fermenting organism;
wherein the alpha-amylase has at least 60%, at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 29 or SEQ ID NO: 30.

11. A raw starch hydrolysis process for producing a fermentation product from a raw starch material comprising the steps of:
(a) saccharifying a raw starch material at a temperature below the initial gelatinization temperature of said raw starch material using a glucoamylase and an alpha-amylase; and
(b) fermenting with a fermenting organism;
wherein the alpha-amylase has at least 60%, at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 32 or SEQ ID NO: 33.

12. A raw starch hydrolysis process for producing a fermentation product from a raw starch material comprising the steps of:
(a) saccharifying a raw starch material at a temperature below the initial gelatinization temperature of said raw starch material using a glucoamylase and an alpha-amylase; and
(b) fermenting with a fermenting organism;
wherein the alpha-amylase has at least 60%, at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 35 or SEQ ID NO: 36.

13. A raw starch hydrolysis process for producing a fermentation product from a raw starch material comprising the steps of:
(a) saccharifying a raw starch material at a temperature below the initial gelatinization temperature of said raw starch material using a glucoamylase and an alpha-amylase; and
(b) fermenting with a fermenting organism;
wherein the alpha-amylase has at least 60%, at least 65%, at least 70%, at least 75%, at least 80%, at least 85%, at least 90%, at least 95%, at least 96%, at least 97%, at least 98%, or at least 99% sequence identity, or 100% sequence identity, to the polypeptide of SEQ ID NO: 38 or SEQ ID NO: 39.

14. The raw starch hydrolysis process of claim 1, wherein steps (a) and (b) are carried out simultaneously.

15. The raw starch hydrolysis process of claim 1, wherein the fermenting organism expresses the alpha-amylase in situ during fermentation.

16. The raw starch hydrolysis process of claim 1, wherein the fermenting organism expresses the alpha-amylase and the glucoamylase in situ during fermentation.

17. The raw starch hydrolysis process of claim 1, wherein the fermenting organism is a yeast cell.

18. The raw starch hydrolysis process of claim 1, wherein the fermentation product is an alcohol, such as ethanol, preferably fuel ethanol.

19. The raw starch hydrolysis process of claim 1, wherein the glucoamylase comprises the amino acid sequence of SEQ ID NO: 18, or has an amino acid sequence that is at least 70%, at least 75%, at least 80%, at least 85%, at least 90%, at least 95%, at least 96%, at least 97%, at least 98%, or at least 99% identical to SEQ ID NO: 18.


SEQUENCE LISTING (39 sequences)

SEQ ID NO: 1 - DNA, 1812 bp, Artificial Sequence
  Hybrid alpha-amylase Penicillium oxalicum catalyic domain and Aspergillus rolfsii starch binding domain
  CDS (1)..(1812)
  sig_peptide (1)..(60)
  misc_structure (61)..(1479): catalytic domain
  misc_binding (1519)..(1809): starch binding domain
atgaaattccttggactagctgctttgtttcttgcccagaccgtggcgggtctgacggctgcccaatggcgcagccaatcgatctacttcctcatgactgatcggtttggtcgaaccgacaagtccgtgactgccccatgcaacacaaatgaccgagtatactgtggtggaacttggcaaggcattatcaatcaattggattacatccaaggaatgggatttactgcgatttggatcactccagtcacagagcagctccctcaagatactggggacggtgaagcataccacggatattggcaacaggaaatatacaatgtcaacaacaactacggcactgctgccgatctcaaggctctttcgcaagcccttcacagtcgtggaatgtacctcatggttgatgttgtcgcgaaccacatgggctacgcgggggccggaaacaccgtcgactacagtgtgttcaagccattcagctcctcttcgtacttccacccgtattgtttgatcagcgattactcaaatcagacaaatgtcgaggactgttggcttggggacaccaccgtctcactccccgatcttgataccactctttcttccgtgcagacgatctggtataattgggtcagtgacctagtttcaaactattccatcgacggtcttcggatcgatactgtcaagcacgttcaaaagtcattctggcctggctatcagagtgctgctggtgtctactgtgttggagaggtcttcagtggtgatccggcttacacttgcccttatcaaaactaccttgatggggttttgaattatccaatctattaccaattactcggcgcatttaaatcaaccagtggaagcatcagcagcctttacaacatgatcaactctgttgcgtccgactgtgctgacccaaccctgctgggtaactttattgaaaaccatgataacccacgctttgcttcgtacacaagcgactactcgcaagcgaaaaatgtcatctcgttcattttcttgtctgatggcattccgatcgtctatgccggacaggaacagcactacagtggcggtaacgaccctgctaaccgtgaagcgacttggctttccggatactcaaagaacgctcaactgtaccaacacattgcttcgacgaacaaaatccgcagtcttgcgatttcaaaggatgccaactacatcacttccaagaacaatgctttctacactgacagtaacaccatcgccatgaagaaaggatcctccggatcgcaggtcgtgacagttctctcaaaccgtggctcgtcaggtagctcgtacactttgagtctcagcggcagcggctatgcggctggcaccaagctggtggaaatgtacacctgcaccgctgttaccgttgattcgaatggcaacatcgcagtttccatgacctctgggctgcctcgagtgtttatgctggcctcctctgcatgctctctttgcagttccgcggaattcggtgctacaagcccgggtggctcctcgggtagtgtcgaggtcactttcgacgtttacgctaccacagtatatggccagaacatctatatcaccggtgatgtgagtgagctcggcaactggacacccgccaatggtgttgcactctcttctgctaactaccccacctggagtgccacgatcgctctccccgctgacacgacaatccagtacaagtatgtcaacattgacggcagcaccgtcatctgggaggatgctatcagcaatcgcgagatcacgacgcccgccagcggcacatacaccgaaaaagacacttgggatgaatcttag

SEQ ID NO: 2 - protein, 603 aa, Artificial Sequence
  Synthetic Construct
MKFLGLAALFLAQTVAGLTAAQWRSQSIYFLMTDRFGRTDKSVTAPCNTNDRVYCGGTWQGIINQLDYIQGMGFTAIWITPVTEQLPQDTGDGEAYHGYWQQEIYNVNNNYGTAADLKALSQALHSRGMYLMVDVVANHMGYAGAGNTVDYSVFKPFSSSSYFHPYCLISDYSNQTNVEDCWLGDTTVSLPDLDTTLSSVQTIWYNWVSDLVSNYSIDGLRIDTVKHVQKSFWPGYQSAAGVYCVGEVFSGDPAYTCPYQNYLDGVLNYPIYYQLLGAFKSTSGSISSLYNMINSVASDCADPTLLGNFIENHDNPRFASYTSDYSQAKNVISFIFLSDGIPIVYAGQEQHYSGGNDPANREATWLSGYSKNAQLYQHIASTNKIRSLAISKDANYITSKNNAFYTDSNTIAMKKGSSGSQVVTVLSNRGSSGSSYTLSLSGSGYAAGTKLVEMYTCTAVTVDSNGNIAVSMTSGLPRVFMLASSACSLCSSAEFGATSPGGSSGSVEVTFDVYATTVYGQNIYITGDVSELGNWTPANGVALSSANYPTWSATIALPADTTIQYKYVNIDGSTVIWEDAISNREITTPASGTYTEKDTWDES

SEQ ID NO: 3 - protein, 583 aa, Artificial Sequence
  Hybrid alpha-amylase Penicillium oxalicum catalyic domain and Aspergillus rolfsii starch binding domain
AQWRSQSIYFLMTDRFGRTDKSVTAPCNTNDRVYCGGTWQGIINQLDYIQGMGFTAIWITPVTEQLPQDTGDGEAYHGYWQQEIYNVNNNYGTAADLKALSQALHSRGMYLMVDVVANHMGYAGAGNTVDYSVFKPFSSSSYFHPYCLISDYSNQTNVEDCWLGDTTVSLPDLDTTLSSVQTIWYNWVSDLVSNYSIDGLRIDTVKHVQKSFWPGYQSAAGVYCVGEVFSGDPAYTCPYQNYLDGVLNYPIYYQLLGAFKSTSGSISSLYNMINSVASDCADPTLLGNFIENHDNPRFASYTSDYSQAKNVISFIFLSDGIPIVYAGQEQHYSGGNDPANREATWLSGYSKNAQLYQHIASTNKIRSLAISKDANYITSKNNAFYTDSNTIAMKKGSSGSQVVTVLSNRGSSGSSYTLSLSGSGYAAGTKLVEMYTCTAVTVDSNGNIAVSMTSGLPRVFMLASSACSLCSSAEFGATSPGGSSGSVEVTFDVYATTVYGQNIYITGDVSELGNWTPANGVALSSANYPTWSATIALPADTTIQYKYVNIDGSTVIWEDAISNREITTPASGTYTEKDTWDES

SEQ ID NO: 4 - DNA, 2351 bp, Penicillium sclerotiorum
  sig_peptide (1)..(51)
  exon (1)..(156)
  misc_structure (52)..(1943): catalytic domain
  Intron (157)..(220)
  exon (221)..(259)
  Intron (260)..(315)
  exon (316)..(431)
  Intron (432)..(483)
  exon (484)..(592)
  Intron (593)..(648)
  exon (649)..(877)
  Intron (878)..(937)
  exon (938)..(1100)
  Intron (1101)..(1148)
  exon (1149)..(1295)
  Intron (1296)..(1341)
  exon (1342)..(1582)
  Intron (1583)..(1652)
  exon (1653)..(2351)
  misc_binding (2046)..(2348): starch binding domain
atgaatcgagttctattagcttcgacactattccttgcccaggccttgagccttacacccgcggaatggcgcagtcaatctatctactttctgttgacggaccgattcgctcggacggacggttcaactacagcgacatgcgatgtctctgatagagtaggatctgaacttcgttacttgaccagaaacatcaagatcaaatctgacatggcattgatagatctactgcggaggcagctggcaagggataataaatcaggtattattccctttaaatatatacaaggattgattcgaatgctaagattatgctagctggactatatccaagggatgggctttactgccatctggattacccccgtcactgagcaattgtctcaggacacaggagacggtgaggcatatcacggatattggcagcagaagatgtaggagccagttgttaccattgatatatactgctcctactaacttgctcagctacgatgtcaacacgaactacggaacggcagctgatctcttagccttatccgaggccttacatgcccgagacatgtatctaatggtggacgttgttgcaaaccacatggtaagtcctcatttcctgtaaaaaacatgaatcatctcttcttatgcttttaatagggctatgatggagcgggcagtagtgtggactacagcgtctttgatccgttcgattcttcctcttactttcattcttactgcctcatcagtaactatgataaccagaccaatgtcgaggattgctggcttggagatacaacggtctctctgccggatctcaataccgagctgtcctctgttgagaccatatggtatgactggattgcagatctagtctccaactactccagtaagtaatcggtcactaaagaacataatgctgcctggcgagatctgacgttaattacagttgatggacttcgaatcgataccgtgaaacatgtccaagaatccttttggccgggctacaatgatgccgcaggtgtttactgtgttggggaagtttttgatggcgatccggcatacacctgcccttaccagaattatctggacggagtgctgaactacccaatgtaagtccctttccgtctactcttccactaagctaatatattgcaaagttactaccagttgctgtacgctttcgaagattcaagcggaagcatcagtgacctgtacaacatgatcaactcggtcgcttctgactgcgctgatcccacactgcttggaaacttcattgagaaccacgacaacccaagatttgcctcgtaagtactctgaagctttattcggctcttactgactggtgaaaagctacactgatgattactcccaagcgaagaatgtaatctcctttatattcctctctgacggcattccgatcgtctacgcaggccaggagcagcactacagcggcggtgctgacccctataaccgagaagcaacttggctgtcgggatactctaccactgctgagctgtacgagtacatcgcaacgaccaacaaaatccgcagcctcgccatctcctctgattcctcctacattacttccgaggtaaagtcaacctacaccccattcctcatgaattcacttcgagtcataactaacgttaaggaatattaagaacgatgctttctaccaggatagccacaccttggctatgaagaagggatccagtggctcccaagtcattaccgtgctctccaactacggtgcctctggtagctcatacactttgaccctgagcggtagtgggtattcctcaggtacccagcttatggagttgtacacatgtacctccgtgacagtagactcgagcggcaatatcgccgtacccatggcatctggtctaccccgggtctttatgcttgcctctgctgtatccagtagcagcatttgcggtgggtcgtcatccgctacaacaacatcgtcctcaactaagacatccactacattgacgacgacaacttctaagacgacaactgcgtcttcctgcacccaggccacggctttgcctgttttgtttgaggagatcgtgacgacaacctatggtgaggatatctacatctccggttcgatcagtcagctaggctcgtgggataccgatgatgctattgccttgtctgcggatgagtacacttcatcgaatcatctatggtacgttgttgtcaccattccagtgggaacgtccttccagtacaagtttattgaaaagacggacggttcatcttctgtgagttgggaaagcgatccaaatagatcttactctgtcccgaccggatgcgtgggctctactgcaactgtgacggcaacctggagatga

SEQ ID NO: 5 - protein, 632 aa, Penicillium sclerotiorum
  SIGNAL (1)..(17)
  DOMAIN (18)..(497): catalytic domain
  mat_peptide (18)..(632)
  BINDING (532)..(632): starch binding domain
MNRVLLASTLFLAQALSLTPAEWRSQSIYFLLTDRFARTDGSTTATCDVSDRIYCGGSWQGIINQLDYIQGMGFTAIWITPVTEQLSQDTGDGEAYHGYWQQKIYDVNTNYGTAADLLALSEALHARDMYLMVDVVANHMGYDGAGSSVDYSVFDPFDSSSYFHSYCLISNYDNQTNVEDCWLGDTTVSLPDLNTELSSVETIWYDWIADLVSNYSIDGLRIDTVKHVQESFWPGYNDAAGVYCVGEVFDGDPAYTCPYQNYLDGVLNYPIYYQLLYAFEDSSGSISDLYNMINSVASDCADPTLLGNFIENHDNPRFASYTDDYSQAKNVISFIFLSDGIPIVYAGQEQHYSGGADPYNREATWLSGYSTTAELYEYIATTNKIRSLAISSDSSYITSENDAFYQDSHTLAMKKGSSGSQVITVLSNYGASGSSYTLTLSGSGYSSGTQLMELYTCTSVTVDSSGNIAVPMASGLPRVFMLASAVSSSSICGGSSSATTTSSSTKTSTTLTTTTSKTTTASSCTQATALPVLFEEIVTTTYGEDIYISGSISQLGSWDTDDAIALSADEYTSSNHLWYVVVTIPVGTSFQYKFIEKTDGSSSVSWESDPNRSYSVPTGCVGSTATVTATWR

SEQ ID NO: 6 - protein, 615 aa, Penicillium sclerotiorum
LTPAEWRSQSIYFLLTDRFARTDGSTTATCDVSDRIYCGGSWQGIINQLDYIQGMGFTAIWITPVTEQLSQDTGDGEAYHGYWQQKIYDVNTNYGTAADLLALSEALHARDMYLMVDVVANHMGYDGAGSSVDYSVFDPFDSSSYFHSYCLISNYDNQTNVEDCWLGDTTVSLPDLNTELSSVETIWYDWIADLVSNYSIDGLRIDTVKHVQESFWPGYNDAAGVYCVGEVFDGDPAYTCPYQNYLDGVLNYPIYYQLLYAFEDSSGSISDLYNMINSVASDCADPTLLGNFIENHDNPRFASYTDDYSQAKNVISFIFLSDGIPIVYAGQEQHYSGGADPYNREATWLSGYSTTAELYEYIATTNKIRSLAISSDSSYITSENDAFYQDSHTLAMKKGSSGSQVITVLSNYGASGSSYTLTLSGSGYSSGTQLMELYTCTSVTVDSSGNIAVPMASGLPRVFMLASAVSSSSICGGSSSATTTSSSTKTSTTLTTTTSKTTTASSCTQATALPVLFEEIVTTTYGEDIYISGSISQLGSWDTDDAIALSADEYTSSNHLWYVVVTIPVGTSFQYKFIEKTDGSSSVSWESDPNRSYSVPTGCVGSTATVTATWR

SEQ ID NO: 7 - DNA, 2339 bp, Penicillium wotroi
  sig_peptide (1)..(63)
  exon (1)..(156)
  misc_structure (64)..(1925): Catalytic Domain
  Intron (157)..(215)
  exon (216)..(254)
  Intron (255)..(307)
  exon (308)..(423)
  Intron (424)..(478)
  exon (479)..(587)
  Intron (588)..(638)
  exon (639)..(867)
  Intron (868)..(929)
  exon (930)..(1092)
  Intron (1093)..(1149)
  exon (1150)..(1296)
  Intron (1297)..(1347)
  exon (1348)..(1588)
  Intron (1589)..(1640)
  exon (1641)..(2339)
  misc_binding (2034)..(2336): starch binding domain
atgaaagcacttgcattgaccgcggtgttgcttacgaaggctgtcgtcggcctgacgcctgcagaatggcgaagtcagtcgatttactttcttctgactgatcgatttgggcgtacagataattctacaacagcaacatgcaatgttagcgaccgtgtaaggactcaaatacccccttccccggtacagctcgtattactaaatgactgcattagatttactgcggtggtagctggcaaggaatcatcaaccacgtacgctgctcttccacctcctaattctcaagcactgctaagtctccgtcaagttggactatattcaagacatgggattcactgctatttggattacccccgtcacagaacagctttctcaagatactggagatggcgaagcatatcatggatactggcaacaggagatgttcgtgggctctgttgaccctgaagctacgattgaactgacgcttttttattagatacaatgtcaacacgaactatggcactgcttctgatcttttggcactctctgaagctctacatagccgtggcatgtatctcatggtagatgtggttgcaaaccatatggtatgcgctctattcataacaaagcgatctggtctctgactaaatatacagggatacgatggagctggtaacactgttgactacagtgttttcaacccatttgactcttcctcttacttccattcgtattgtgagatcagcgattactctaaccagacaaacgtggaagactgttggctaggagacactacggtctcgcttcccgatcttgacacgacactttcttctgttcaaacaatctggtatgactgggtggccgctttggtgtcgaattattcaagtatgtacaaattgcgaggtattttacaaagtcctgagatccgctaacatactacgacaaagttgatggattacgagttgatacggtcaagcacgtccagaagtcattttggcccggctacaatagtgcggcaggcgtctactgtgttggagaggtctttgatggagacccagcatacacatgcccttaccaggactacatggatggcgttttgaactatccgatgtatgttttcacccctgggactgttattttgatggatgttttcctgacctacaccagttattatcaactgctgtatgcattcgagtcaacaagcggcagtatcagcaacctatacaatatgatcagctccgttgcgtctgattgctctgacactaccttgctgggaaacttcattgaaaatcatgacaacccacgatttgcttcgtatgacccccaagtgtggattaacgttcgctactaacacctccactttagctacacaagtgactactctcaagcgaagaatgtgctttctttcatcttcttctctgatggaatccctatcgtctatgctggccaggaacaacactatagcggcggtagcgatccggctaatcgtgaggcaacctggctatccggatacgatacaagcgcggaactctacacgtggattgcctccaccaacaagatccgcagtctggctatctctaaggatactgcctacgtaacatccaaggtacgcattggcaacttatgtcttatacgacttaactaacaacagcatgcagaatgatgcattctacaccgacagcagtaccatcgccatgaagaaaggatcgagtggctcacaagtcatcactgttctgtcaaaccttggctcatcgggtagctcgtataccttgactctcagtggaagcggctattcatccggcacgaagctgatggagatgtacacctgcactgccgttactgtggactctagtggtggcattgcggtacccatggcttctgggttgcctcgagtatatatgctggcttcttcggcttgttcgatctgtagttctgcctgctcgacgactacaacttcatcgacgacctcgactgcgacaacgacgtcaaccactcttaaaactactacatctaccacctctacgagctgtacacaggctactgctttgcctgttctgtttcaagaaattgttaccacttcatacggacaaaacatctatatctcgggctcgatcagtgagctcggtgactgggacacttctaacgccgttgccctctctgccaacaagtacacatcatccgatcatctttggtatgttgttgtgacaattcctgtgggaacgtcattcgagtacaagtttatcgaggagacgagtgggtctagtagtatcacttgggagagtgatcctaaccgctcgtacacggtcccaactggctgtgccggctcaacagcaaccgttacggcaacatggagatag

SEQ ID NO: 8 - protein, 632 aa, Penicillium wotroi
  SIGNAL (1)..(21)
  DOMAIN (22)..(495): catalytic domain
  mat_peptide (22)..(632)
  BINDING (532)..(632): starch binding module
MKALALTAVLLTKAVVGLTPAEWRSQSIYFLLTDRFGRTDNSTTATCNVSDRIYCGGSWQGIINHLDYIQDMGFTAIWITPVTEQLSQDTGDGEAYHGYWQQEIYNVNTNYGTASDLLALSEALHSRGMYLMVDVVANHMGYDGAGNTVDYSVFNPFDSSSYFHSYCEISDYSNQTNVEDCWLGDTTVSLPDLDTTLSSVQTIWYDWVAALVSNYSIDGLRVDTVKHVQKSFWPGYNSAAGVYCVGEVFDGDPAYTCPYQDYMDGVLNYPIYYQLLYAFESTSGSISNLYNMISSVASDCSDTTLLGNFIENHDNPRFASYTSDYSQAKNVLSFIFFSDGIPIVYAGQEQHYSGGSDPANREATWLSGYDTSAELYTWIASTNKIRSLAISKDTAYVTSKNDAFYTDSSTIAMKKGSSGSQVITVLSNLGSSGSSYTLTLSGSGYSSGTKLMEMYTCTAVTVDSSGGIAVPMASGLPRVYMLASSACSICSSACSTTTTSSTTSTATTTSTTLKTTTSTTSTSCTQATALPVLFQEIVTTSYGQNIYISGSISELGDWDTSNAVALSANKYTSSDHLWYVVVTIPVGTSFEYKFIEETSGSSSITWESDPNRSYTVPTGCAGSTATVTATWR

SEQ ID NO: 9 - protein, 611 aa, Penicillium wotroi
EWRSQSIYFLLTDRFGRTDNSTTATCNVSDRIYCGGSWQGIINHLDYIQDMGFTAIWITPVTEQLSQDTGDGEAYHGYWQQEIYNVNTNYGTASDLLALSEALHSRGMYLMVDVVANHMGYDGAGNTVDYSVFNPFDSSSYFHSYCEISDYSNQTNVEDCWLGDTTVSLPDLDTTLSSVQTIWYDWVAALVSNYSIDGLRVDTVKHVQKSFWPGYNSAAGVYCVGEVFDGDPAYTCPYQDYMDGVLNYPIYYQLLYAFESTSGSISNLYNMISSVASDCSDTTLLGNFIENHDNPRFASYTSDYSQAKNVLSFIFFSDGIPIVYAGQEQHYSGGSDPANREATWLSGYDTSAELYTWIASTNKIRSLAISKDTAYVTSKNDAFYTDSSTIAMKKGSSGSQVITVLSNLGSSGSSYTLTLSGSGYSSGTKLMEMYTCTAVTVDSSGGIAVPMASGLPRVYMLASSACSICSSACSTTTTSSTTSTATTTSTTLKTTTSTTSTSCTQATALPVLFQEIVTTSYGQNIYISGSISELGDWDTSNAVALSANKYTSSDHLWYVVVTIPVGTSFEYKFIEETSGSSSITWESDPNRSYTVPTGCAGSTATVTATWR

SEQ ID NO: 10 - DNA, 1830 bp, Talaromyces helicus
  CDS (1)..(1830)
  sig_peptide (1)..(57)
  misc_structure (58)..(1488): catalytic domain
  misc_binding (1504)..(1827): starch binding domain
atgaaactgtcaaccctttccctcctctggggctatgtcgccaacaccgctctcggactcagcgccagcggctggcgcagccagtccatctacttcctcatgaccgaccggtttggtcttaccaacggctctacgactgctccgtgcaatgctgccgatggcatttactgcggtggcagctggcagggcgtcattaaccatttggactatatccagggaatgggcttcaccgcactatggatcaccccagtcaccgagaacttcttgggcaacacacaggacggtgagagttaccacggatactggcagcaaaacgcctatgcagtcaacgcccagttcgggagcagctccgaccttctcgcgctgtcgaaggcgctgcatgcccgtggcatgtatttgatggttgacatcgtggtcaataacatggcgtacgatggtcctggcacaaacgtcgattactccattttcaacccattcccctccgagtcctactaccatccgtactgcctcatcagcaattacaacaaccagaccaacgtggaggactgttgggagggcgacacaattgtttcgcttcccgatttggacaccacgcagacctacgtgcaggacacctggaatgcctgggtgaagagcttcgtctccaactactcgatcgatggcctgaggattgactccgctaaacacatgcaaaaagactttttccccaatttcgaggattccgcaaatgtgtactgcataggggaagttcttgatggtgacccgacttatgtgtgcccgtatcaggattatctgaacggcgtgttgaactaccctatctactaccaactgctctacgcatttgagtcctcgtccggatccatctccaacctctacgacatgattaacagcgtcaagagcgattgtgccgacagtacccttcttggcaatttcgccgagaaccacgacaatcctcgtttcgcccattacacctcggacttcagccaggctaagtcggtgatcagcttcattttccttacagacggcatcccaatcctctactatggccaagagcagcacttcagtggtggcaatgttccgctcaaccgcgagcctctctggcccagcgactattcgacgaccgctgagctctacacccatactgcgacgaccaatgctatccgaacgcttgctatgaagaaggacgcaggttttctcacatacaagaataccccgttctaccaagattccaataccatcgcgatgcgcaaaggtaccactggactccagctcatcactgtactttcgaacctaggttcctcggggtcgtcgtacacgctgtcgctatcaggctccgggtacacttccggcacgcttctcactgagctctacacgtgtaccaacgttacagtatcctcatccggcacgattgctgtccccatgtcctctggcgaacctcgcgctttcctgccttggtccagcgtgtcaggcagctccctctgccagggaactgctgtgagctcctgtaccactgcgtcttctgtcgcggtcactttcaatgagctcgtcaccacttcttacggccaggaggtataccttgccggttccatctctcagctggggagctggtccacctcgaacgccgtgttgctctccgctagcaagtatacttcctccaaccctttgtggttcgtcaccgtgaaccttcccgctggcactacattccagtacaagttcatcatcaagaacaccgacggttccttcacttgggaaagcgatcccaaccgatcctacaccgtcccaacgggctgccagggcctcactgccacagtgaacgacacctggcgctaa

SEQ ID NO: 11 - protein, 609 aa, Talaromyces helicus
MKLSTLSLLWGYVANTALGLSASGWRSQSIYFLMTDRFGLTNGSTTAPCNAADGIYCGGSWQGVINHLDYIQGMGFTALWITPVTENFLGNTQDGESYHGYWQQNAYAVNAQFGSSSDLLALSKALHARGMYLMVDIVVNNMAYDGPGTNVDYSIFNPFPSESYYHPYCLISNYNNQTNVEDCWEGDTIVSLPDLDTTQTYVQDTWNAWVKSFVSNYSIDGLRIDSAKHMQKDFFPNFEDSANVYCIGEVLDGDPTYVCPYQDYLNGVLNYPIYYQLLYAFESSSGSISNLYDMINSVKSDCADSTLLGNFAENHDNPRFAHYTSDFSQAKSVISFIFLTDGIPILYYGQEQHFSGGNVPLNREPLWPSDYSTTAELYTHTATTNAIRTLAMKKDAGFLTYKNTPFYQDSNTIAMRKGTTGLQLITVLSNLGSSGSSYTLSLSGSGYTSGTLLTELYTCTNVTVSSSGTIAVPMSSGEPRAFLPWSSVSGSSLCQGTAVSSCTTASSVAVTFNELVTTSYGQEVYLAGSISQLGSWSTSNAVLLSASKYTSSNPLWFVTVNLPAGTTFQYKFIIKNTDGSFTWESDPNRSYTVPTGCQGLTATVNDTWR

SEQ ID NO: 12 - protein, 590 aa, Talaromyces helicus
LSASGWRSQSIYFLMTDRFGLTNGSTTAPCNAADGIYCGGSWQGVINHLDYIQGMGFTALWITPVTENFLGNTQDGESYHGYWQQNAYAVNAQFGSSSDLLALSKALHARGMYLMVDIVVNNMAYDGPGTNVDYSIFNPFPSESYYHPYCLISNYNNQTNVEDCWEGDTIVSLPDLDTTQTYVQDTWNAWVKSFVSNYSIDGLRIDSAKHMQKDFFPNFEDSANVYCIGEVLDGDPTYVCPYQDYLNGVLNYPIYYQLLYAFESSSGSISNLYDMINSVKSDCADSTLLGNFAENHDNPRFAHYTSDFSQAKSVISFIFLTDGIPILYYGQEQHFSGGNVPLNREPLWPSDYSTTAELYTHTATTNAIRTLAMKKDAGFLTYKNTPFYQDSNTIAMRKGTTGLQLITVLSNLGSSGSSYTLSLSGSGYTSGTLLTELYTCTNVTVSSSGTIAVPMSSGEPRAFLPWSSVSGSSLCQGTAVSSCTTASSVAVTFNELVTTSYGQEVYLAGSISQLGSWSTSNAVLLSASKYTSSNPLWFVTVNLPAGTTFQYKFIIKNTDGSFTWESDPNRSYTVPTGCQGLTATVNDTWR

SEQ ID NO: 13 - DNA, 1428 bp, Lactobacillus amylovorus
  CDS (1)..(1428)
  sig_peptide (1)..(87)
  misc_structure (88)..(1407): catalytic domain
  misc_structure (1408)..(1425): His-tag
atgaaacaacaaaaacggctttacgcccgattgctgacgctgttatttgcgctcatcttcttgctgcctcattctgcagcagcggcggctagtgatacgacatcaactgatcactcaagcaatgatacagctgattctgttagcgacggtgttattttgcatgcatggtgctggtcgttcaacacgattaaaaacaacttgaaacagattcatgacgccggctacacagcggttcaaacttcacctgttaatgaagttaaagttggaaatagcgggtctaagtcattaaataactggtattggctatatcagccaactaaatatagtattggtaactattatttaggaacggaagctgaatttaagtcaatgtgcgctgctgctaaagaatataatatcaggatcattgtcgatgcaactctgaatgatacaacaagtgattatagtgcaatttcggatgaaattaaaagtattccaaattggacacatggtaacaaacaaatttcgaattggagtgatcgtgaagatgttactcaaaattcgttgttaggtttatatgattggaatactcaaaattctcaagtccagacgtatttgaagaattatttggaacgcttgatttctgacggagcttcaggctttcgttatgatgcagctaagcatattgaacttccaagtcaatatgatggcagctatggcagcaatttctggccaaatattactgataatgggtctgaatttcagtatggtgaagttttgcaggactcgatttcaaaagaatcagattatgctaattacatgagtgttacagcttcaaattacggcaatacgattcgcaatgcgttaaagaatcgtgattttaccgcaagtactttgcagaatttcaacatcagtgttccagcttctaaattagtaacttgggtcgaatcgcatgataattatgctaacgatgatcaagtttcgacttggatgaataatagtgatattaaattaggctgggctgttgttgcttcgcgttctggtagtgttccgctgttctttgaccgtccagttgatggtggtaatggtactcggttccctggcagttcagaaattggtgatgctggcagcagtttgtattatgataaagcagttgtagctgttaataaattccataatgcaatggctggtcaatctgaatatatttctaatccaaatggcaataccaagatttttgaaaatgaacgtggcagcaaaggggttgtttttgcaaacgcttccgacagttcatatagtttgaatgttaaaactagtttagctgatgggacttatgaaaacaaggctggttcagatgaacttaccgttaaaaatggttatttaaccggtacaattcaaggacgtgaagttgttgttctttacggggatccacatcatcaccatcaccactaa

SEQ ID NO: 14 - protein, 475 aa, Lactobacillus amylovorus
MKQQKRLYARLLTLLFALIFLLPHSAAAAASDTTSTDHSSNDTADSVSDGVILHAWCWSFNTIKNNLKQIHDAGYTAVQTSPVNEVKVGNSGSKSLNNWYWLYQPTKYSIGNYYLGTEAEFKSMCAAAKEYNIRIIVDATLNDTTSDYSAISDEIKSIPNWTHGNKQISNWSDREDVTQNSLLGLYDWNTQNSQVQTYLKNYLERLISDGASGFRYDAAKHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNNSDIKLGWAVVASRSGSVPLFFDRPVDGGNGTRFPGSSEIGDAGSSLYYDKAVVAVNKFHNAMAGQSEYISNPNGNTKIFENERGSKGVVFANASDSSYSLNVKTSLADGTYENKAGSDELTVKNGYLTGTIQGREVVVLYGDPHHHHHH

SEQ ID NO: 15 - protein, 446 aa, Lactobacillus amylovorus
ASDTTSTDHSSNDTADSVSDGVILHAWCWSFNTIKNNLKQIHDAGYTAVQTSPVNEVKVGNSGSKSLNNWYWLYQPTKYSIGNYYLGTEAEFKSMCAAAKEYNIRIIVDATLNDTTSDYSAISDEIKSIPNWTHGNKQISNWSDREDVTQNSLLGLYDWNTQNSQVQTYLKNYLERLISDGASGFRYDAAKHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNNSDIKLGWAVVASRSGSVPLFFDRPVDGGNGTRFPGSSEIGDAGSSLYYDKAVVAVNKFHNAMAGQSEYISNPNGNTKIFENERGSKGVVFANASDSSYSLNVKTSLADGTYENKAGSDELTVKNGYLTGTIQGREVVVLYGDPHHHHHH

SEQ ID NO: 16 - protein, 586 aa, Valsaria rubricosa
  SIGNAL (1)..(20)
  mat_peptide (21)..(586)
  DOMAIN (21)..(460): catalytic domain
  BINDING (483)..(586): starch binding domain
MRSFLALSALLLLYPLQLLAASNSDWRSRNIYFALTDRVANPSTTTACSDLSNYCGGTWSGLSSKLDYIQGMGFDSIWITPVVENCDGGYHGYWAKALYNVNTNYGSADDLKNFVAAAHAKGMYVMVDVVANHMGSCGIANLSPPPLNEQSSYHTQCDIDYSSQSSIETCWISGLPDLDTTDSTIRSLFQTWVHGLVSNYSFDGLRVDTVKHVEKDYWPGFVSAAGTYAIGEVFSGDTSYVAGYQSVMPGLLNYPIYYPLIRVFAQGASFTDLVNNHDTVGSTFSDPTLLGNFIDNHDNPRFLSYTSDHALLKNALAYVILARGIPIVYYGTEQGYSGSSDPANREDLWRSGYSTTGDIYTTIAALSAARTAAGGLAGNDHVHLYTTDNAYAWSRASGKLIVVTSNRGSSDSSTICFSTQQASGTTWTSTITGNSYTADSNGQICVQLSSGGPEALVVSTATGTATATTLSTTTKTSTSTASCAATVAVTFNELVTTNYGDTIRLTGSISQLSSWSATSGLALSASAYTSSNPLWSVTVSLPAGTSFEYKFVRITSDGTVTWESDPNRSYTVPTCASTATISNTWR

SEQ ID NO: 17 - protein, 566 aa, Valsaria rubricosa
ASNSDWRSRNIYFALTDRVANPSTTTACSDLSNYCGGTWSGLSSKLDYIQGMGFDSIWITPVVENCDGGYHGYWAKALYNVNTNYGSADDLKNFVAAAHAKGMYVMVDVVANHMGSCGIANLSPPPLNEQSSYHTQCDIDYSSQSSIETCWISGLPDLDTTDSTIRSLFQTWVHGLVSNYSFDGLRVDTVKHVEKDYWPGFVSAAGTYAIGEVFSGDTSYVAGYQSVMPGLLNYPIYYPLIRVFAQGASFTDLVNNHDTVGSTFSDPTLLGNFIDNHDNPRFLSYTSDHALLKNALAYVILARGIPIVYYGTEQGYSGSSDPANREDLWRSGYSTTGDIYTTIAALSAARTAAGGLAGNDHVHLYTTDNAYAWSRASGKLIVVTSNRGSSDSSTICFSTQQASGTTWTSTITGNSYTADSNGQICVQLSSGGPEALVVSTATGTATATTLSTTTKTSTSTASCAATVAVTFNELVTTNYGDTIRLTGSISQLSSWSATSGLALSASAYTSSNPLWSVTVSLPAGTSFEYKFVRITSDGTVTWESDPNRSYTVPTCASTATISNTWR

SEQ ID NO: 18 - protein, 556 aa, Trametes cingulata
QSSAADAYVASESPIAKAGVLANIGPSGSKSNGAKAGIVIASPSTSNPNYLYTWTRDSSLVFKALIDQFTTGEDTSLRTLIDEFTSAEAILQQVPNPSGTVSTGGLGEPKFNIDETAFTDAWGRPQRDGPALRATAIITYANWLLDNKNTTYVTNTLWPIIKLDLDYVASNWNQSTFDLWEEINSSSFFTTAVQHRALREGATFANRIGQTSVVSGYTTQANNLLCFLQSYWNPTGGYITANTGGGRSGKDANTVLTSIHTFDPAAGCDAVTFQPCSDKALSNLKVYVDAFRSIYSINSGIASNAAVATGRYPEDSYMGGNPWYLTTSAVAEQLYDALIVWNKLGALNVTSTSLPFFQQFSSGVTVGTYASSSSTFKTLTSAIKTFADGFLAVNAKYTPSNGGLAEQYSRSNGSPVSAVDLTWSYAAALTSFAARSGKTYASWGAAGLTVPTTCSGSGGAGTVAVTFNVQATTVFGENIYITGSVPALQNWSPDNALILSAANYPTWSITVNLPASTTIEYKYIRKFNGAVTWESDPNNSITTPASGTFTQNDTWR

SEQ ID NO: 19 - DNA, 1980 bp, Bacillus amyloliquefaciens
  CDS (1)..(1977)
  sig_peptide (1)..(81)
  mat_peptide (82)..(1977)
  misc_structure (82)..(1395): catalytic domain
  misc_binding (1660)..(1959): starch binding domain
  misc_structure (1960)..(1977): His-tag
atgaaaaaaccgctggggaaaattgtcgcaagcaccgcactactcatttctgttgcttttagttcatcgatcgcatcggctgaaactgcaaacaaatcgaataaggtgaccgcgtcatcggtcaaaaacgggaccatcctccatgcatggaattggtcgttcaatacgttaacacaaaatatgaaagatattcgtgatgcgggctatgcagccattcagacgtctccgattaaccaagtaaaggaagggaaccaaggagataaaagcatgaggaactggtactggctgtatcagccgacatcgtaccaaatcggcaaccgttacttaggcactgaacaagaatttaaggacatgtgtgcagccgcggaaaaatatggcgtaaaagtcattgtcgatgcggttatcaatcatactaccagcgattatggcgcgatttctgacgagattaagcgtattccaaactggacccatggaaacacacaaattaaaaattggtcggatcgatgggacgtcactcaaaattcattgcttgggctgtatgattggaatacacagaatactgaggtgcaggcatatctgaaacgtttcttggaaagagcattgaatgacggagcagacgggttccgctatgatgccgccaagcatatagagcttccggatgatgggaattacggcagccaattttggccgaatatcacaaatacatcggcggagttccaatacggagaaatcctgcaagacagcgcgtccagagatacagcttatgcgaattatatgaatgtgacggcttctaactacgggcattccatcagatccgctttaaagaaccgtaatctgagtgtgtcgaatatctcccattatgcatctgacgtgtctgcggacaagttagtcacatgggtggaatcacatgatacgtatgccaatgatgatgaagagtccacatggatgagtgatgacgatattcgtttaggctgggcagtgattggttcccgctcaggaagcacgcctcttttcttttccagacctgagggcggaggaaatggtgtaagatttcccggaaaaagtcaaataggagatcgcgggagcgccttatttaaagatcaggcgatcactgcggtcaatacatttcacaatgtaatggccgggcagcctgaggaactctcgaatccgaatgggaacaaccaaatctttatgaatcagcgcggctcaaaaggcgttgtgctggcaaatgcgggatcgtcttctgtcaccatcaatacttcagcgaaattacctgacggcaggtatgataatagggccggcgccggttcatttcaagtagcgaacggcaaactgacaggtacgatcaatgccagatcggcggctgttctttatcctgatgatattggaaatgcgcctcatgtctttcttgagaattaccaaacgggggcagtccattctttcaatgatcagctgacggtcaccctgcgtgcaaatgcgaaaacaacaaaagccgtttaccaaatcaataatgggcagcagacagcatttaaggatggagaccgactaacgatcgggaaaggagatccaatcggcacgacatacaacatcaaattaaccggaacgaacggcgagggtgcagcgaggacccaagaatacaagtttgttaaaaaagacccgtcccaaaccaacatcattggctatcaaaatccggaccattggggccaggtaaatgcttatatttacaaacatgatggaggcagggccatagaattaaccggatcgtggccggggaaagccatgactaagaatgcagatggaatgtacacgctgacgctgcctgagaatgcggatacggccaacgccaaagtgatttttaacaatggcagcgcccaagtgcccggacagaaccagcccggctttgattatgtgcagaatggtttgtataacaactccggtttaaacggttatcttccgcatcatcatcaccatcaccactaa

SEQ ID NO: 20 - protein, 659 aa, Bacillus amyloliquefaciens
MKKPLGKIVASTALLISVAFSSSIASAETANKSNKVTASSVKNGTILHAWNWSFNTLTQNMKDIRDAGYAAIQTSPINQVKEGNQGDKSMRNWYWLYQPTSYQIGNRYLGTEQEFKDMCAAAEKYGVKVIVDAVINHTTSDYGAISDEIKRIPNWTHGNTQIKNWSDRWDVTQNSLLGLYDWNTQNTEVQAYLKRFLERALNDGADGFRYDAAKHIELPDDGNYGSQFWPNITNTSAEFQYGEILQDSASRDTAYANYMNVTASNYGHSIRSALKNRNLSVSNISHYASDVSADKLVTWVESHDTYANDDEESTWMSDDDIRLGWAVIGSRSGSTPLFFSRPEGGGNGVRFPGKSQIGDRGSALFKDQAITAVNTFHNVMAGQPEELSNPNGNNQIFMNQRGSKGVVLANAGSSSVTINTSAKLPDGRYDNRAGAGSFQVANGKLTGTINARSAAVLYPDDIGNAPHVFLENYQTGAVHSFNDQLTVTLRANAKTTKAVYQINNGQQTAFKDGDRLTIGKGDPIGTTYNIKLTGTNGEGAARTQEYKFVKKDPSQTNIIGYQNPDHWGQVNAYIYKHDGGRAIELTGSWPGKAMTKNADGMYTLTLPENADTANAKVIFNNGSAQVPGQNQPGFDYVQNGLYNNSGLNGYLPHHHHHHH

SEQ ID NO: 21 - protein, 633 aa, Bacillus amyloliquefaciens
AETANKSNKVTASSVKNGTILHAWNWSFNTLTQNMKDIRDAGYAAIQTSPINQVKEGNQGDKSMRNWYWLYQPTSYQIGNRYLGTEQEFKDMCAAAEKYGVKVIVDAVINHTTSDYGAISDEIKRIPNWTHGNTQIKNWSDRWDVTQNSLLGLYDWNTQNTEVQAYLKRFLERALNDGADGFRYDAAKHIELPDDGNYGSQFWPNITNTSAEFQYGEILQDSASRDTAYANYMNVTASNYGHSIRSALKNRNLSVSNISHYASDVSADKLVTWVESHDTYANDDEESTWMSDDDIRLGWAVIGSRSGSTPLFFSRPEGGGNGVRFPGKSQIGDRGSALFKDQAITAVNTFHNVMAGQPEELSNPNGNNQIFMNQRGSKGVVLANAGSSSVTINTSAKLPDGRYDNRAGAGSFQVANGKLTGTINARSAAVLYPDDIGNAPHVFLENYQTGAVHSFNDQLTVTLRANAKTTKAVYQINNGQQTAFKDGDRLTIGKGDPIGTTYNIKLTGTNGEGAARTQEYKFVKKDPSQTNIIGYQNPDHWGQVNAYIYKHDGGRAIELTGSWPGKAMTKNADGMYTLTLPENADTANAKVIFNNGSAQVPGQNQPGFDYVQNGLYNNSGLNGYLPHHHHHHH

SEQ ID NO: 22 - DNA, 1725 bp, Lactobacillus amylovorus
  sig_peptide (1)..(81)
  misc_structure (82)..(1725): mature polypeptide coding sequence
atgaagaaaccgttggggaaaattgtcgcaagcaccgcactactcatttctgttgcttttagttcatcgatagcatcagcagctagtgatacgacatcaactgatcactcaagcaatgatacagctgattctgttagcgacggtgttattttgcatgcatggtgctggtcgttcaacacgattaaaaacaacttgaaacagattcatgacgccggctacacagcggttcaaacttcacctgttaatgaagttaaagttggaaatagcgggtctaagtcattaaataactggtattggctatatcagccaactaaatatagtattggtaactattatttaggaacggaagctgaatttaagtcaatgtgcgctgctgctaaagaatataatatcaggatcattgtcgatgcaactctgaatgatacaacaagtgattatagtgcaatttcggatgaaattaaaagtattccaaattggacacatggtaacaaacaaatttcgaattggagtgatcgtgaagatgttactcaaaattcgttgttaggtttatatgattggaatactcaaaattctcaagtccagacgtatttgaagaattatttggaacgcttgatttctgacggagcttcaggctttcgttatgatgcagctaagcatattgaacttccaagtcaatatgatggcagctatggcagcaatttctggccaaatattactgataatgggtctgaatttcagtatggtgaagttttgcaggactcgatttcaaaagaatcagattatgctaattacatgagtgttacagcttcaaattacggcaatacgattcgcaatgcgttaaagaatcgtgattttaccgcaagtactttgcagaatttcaacatcagtgttccagcttctaaattagtaacttgggtcgaatcgcatgataattatgctaacgatgatcaagtttcgacttggatgaataatagtgatattaaattaggctgggctgttgttgcttcgcgttctggtagtgttccgctgttctttgaccgtccagttgatggtggtaatggtactcggttccctggcagttcagaaattggtgatgctggcagcagtttgtattatgataaagcagttgtagctgttaataaattccataatgcaatggctggtcaatctgaatatatttctaatccaaatggcaataccaagatttttgaaaatgaacgtggcagcaaaggggttgtttttgcaaacgcttccgacagttcatatagtttgaatgttaaaactagtttagctgatgggacttatgaaaacaaggctggttcagatgaacttaccgttaaaaatggttatttaaccggtacaattcaaggacgtgaagttgttgttctttacggggatccaacgagcagtagcagcacaacaacagaaacgaaaaaggtgtactttgaaaaaccaagcagttggggaagcagagtttacgcgtatgtctacaataagaacacaaataaagcaattacctcggcttggccgggcaaaaaaatgacggcgcttggaaatgataaatacgaattagatctggacaccgatgaagatgactccgatttagcggtcattttcaccgatgggacaaaacaaacgccggccgctaatgaagcaggcttcacattcacagcggatgcgacgtacgatcaaaatggcgttgtgaaacaccatcaccatcatcac

SEQ ID NO: 23 - protein, 575 aa, Lactobacillus amylovorus
MKKPLGKIVASTALLISVAFSSSIASAASDTTSTDHSSNDTADSVSDGVILHAWCWSFNTIKNNLKQIHDAGYTAVQTSPVNEVKVGNSGSKSLNNWYWLYQPTKYSIGNYYLGTEAEFKSMCAAAKEYNIRIIVDATLNDTTSDYSAISDEIKSIPNWTHGNKQISNWSDREDVTQNSLLGLYDWNTQNSQVQTYLKNYLERLISDGASGFRYDAAKHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNNSDIKLGWAVVASRSGSVPLFFDRPVDGGNGTRFPGSSEIGDAGSSLYYDKAVVAVNKFHNAMAGQSEYISNPNGNTKIFENERGSKGVVFANASDSSYSLNVKTSLADGTYENKAGSDELTVKNGYLTGTIQGREVVVLYGDPTSSSSTTTETKKVYFEKPSSWGSRVYAYVYNKNTNKAITSAWPGKKMTALGNDKYELDLDTDEDDSDLAVIFTDGTKQTPAANEAGFTFTADATYDQNGVVKHHHHHH

SEQ ID NO: 24 - protein, 548 aa, Lactobacillus amylovorus
ASDTTSTDHSSNDTADSVSDGVILHAWCWSFNTIKNNLKQIHDAGYTAVQTSPVNEVKVGNSGSKSLNNWYWLYQPTKYSIGNYYLGTEAEFKSMCAAAKEYNIRIIVDATLNDTTSDYSAISDEIKSIPNWTHGNKQISNWSDREDVTQNSLLGLYDWNTQNSQVQTYLKNYLERLISDGASGFRYDAAKHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNNSDIKLGWAVVASRSGSVPLFFDRPVDGGNGTRFPGSSEIGDAGSSLYYDKAVVAVNKFHNAMAGQSEYISNPNGNTKIFENERGSKGVVFANASDSSYSLNVKTSLADGTYENKAGSDELTVKNGYLTGTIQGREVVVLYGDPTSSSSTTTETKKVYFEKPSSWGSRVYAYVYNKNTNKAITSAWPGKKMTALGNDKYELDLDTDEDDSDLAVIFTDGTKQTPAANEAGFTFTADATYDQNGVVKHHHHHH

SEQ ID NO: 25 - DNA, 1716 bp, Artificial Sequence
  Hybrid comprising Lactobaciilus amylovorus catalytic domain
  sig_peptide (1)..(81)
  misc_structure (82)..(1716): mature polypeptide coding sequence
atgaagaaaccgttggggaaaattgtcgcaagcaccgcactactcatttctgttgcttttagttcatcgatagcatcagcagctagtgatacgacatcaactgatcactcaagcaatgatacagctgattctgttagcgacggtgttattttgcatgcatggtgctggtcgttcaacacgattaaaaacaacttgaaacagattcatgacgccggctacacagcggttcaaacttcacctgttaatgaagttaaagttggaaatagcgggtctaagtcattaaataactggtattggctatatcagccaactaaatatagtattggtaactattatttaggaacggaagctgaatttaagtcaatgtgcgctgctgctaaagaatataatatcaggatcattgtcgatgcaactctgaatgatacaacaagtgattatagtgcaatttcggatgaaattaaaagtattccaaattggacacatggtaacaaacaaatttcgaattggagtgatcgtgaagatgttactcaaaattcgttgttaggtttatatgattggaatactcaaaattctcaagtccagacgtatttgaagaattatttggaacgcttgatttctgacggagcttcaggctttcgttatgatgcagctaagcatattgaacttccaagtcaatatgatggcagctatggcagcaatttctggccaaatattactgataatgggtctgaatttcagtatggtgaagttttgcaggactcgatttcaaaagaatcagattatgctaattacatgagtgttacagcttcaaattacggcaatacgattcgcaatgcgttaaagaatcgtgattttaccgcaagtactttgcagaatttcaacatcagtgttccagcttctaaattagtaacttgggtcgaatcgcatgataattatgctaacgatgatcaagtttcgacttggatgaataatagtgatattaaattaggctgggctgttgttgcttcgcgttctggtagtgttccgctgttctttgaccgtccagttgatggtggtaatggtactcggttccctggcagttcagaaattggtgatgctggcagcagtttgtattatgataaagcagttgtagctgttaataaattccataatgcaatggctggtcaatctgaatatatttctaatccaaatggcaataccaagatttttgaaaatgaacgtggcagcaaaggggttgtttttgcaaacgcttccgacagttcatatagtttgaatgttaaaactagtttagctgatgggacttatgaaaacaaggctggttcagatgaacttaccgttaaaaatggttatttaaccggtacaattcaaggacgtgaagttgttgttctttacggggatccaacttcccaaataacatttactgtaaataacgccacaaccgtttggggacaaaatgtatacgttgtcgggaatatttcgcagctggggaactgggatccagtccacgcagttcaaatgacgccgtcttcttatccaacatggactgtaacaatccctcttcttcaagggcaaaacatacaatttaaatttatcaaaaaagattcagctggaaatgtcatttgggaagatatatcgaatcgaacatacaccgtcccaactgctgcatccggagcatatacagccagctggaacgtgccccaccatcaccatcatcac

SEQ ID NO: 26 - protein, 572 aa, Artificial Sequence
  Hybrid polypeptide comprising Lactobacillus amylovorus catalytic domain
MKKPLGKIVASTALLISVAFSSSIASAASDTTSTDHSSNDTADSVSDGVILHAWCWSFNTIKNNLKQIHDAGYTAVQTSPVNEVKVGNSGSKSLNNWYWLYQPTKYSIGNYYLGTEAEFKSMCAAAKEYNIRIIVDATLNDTTSDYSAISDEIKSIPNWTHGNKQISNWSDREDVTQNSLLGLYDWNTQNSQVQTYLKNYLERLISDGASGFRYDAAKHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNNSDIKLGWAVVASRSGSVPLFFDRPVDGGNGTRFPGSSEIGDAGSSLYYDKAVVAVNKFHNAMAGQSEYISNPNGNTKIFENERGSKGVVFANASDSSYSLNVKTSLADGTYENKAGSDELTVKNGYLTGTIQGREVVVLYGDPTSQITFTVNNATTVWGQNVYVVGNISQLGNWDPVHAVQMTPSSYPTWTVTIPLLQGQNIQFKFIKKDSAGNVIWEDISNRTYTVPTAASGAYTASWNVPHHHHHH

SEQ ID NO: 27 - protein, 545 aa, Artificial Sequence
  Hybrid polypeptide comprising Lactobacillus amylovorus catalytic domain
ASDTTSTDHSSNDTADSVSDGVILHAWCWSFNTIKNNLKQIHDAGYTAVQTSPVNEVKVGNSGSKSLNNWYWLYQPTKYSIGNYYLGTEAEFKSMCAAAKEYNIRIIVDATLNDTTSDYSAISDEIKSIPNWTHGNKQISNWSDREDVTQNSLLGLYDWNTQNSQVQTYLKNYLERLISDGASGFRYDAAKHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNNSDIKLGWAVVASRSGSVPLFFDRPVDGGNGTRFPGSSEIGDAGSSLYYDKAVVAVNKFHNAMAGQSEYISNPNGNTKIFENERGSKGVVFANASDSSYSLNVKTSLADGTYENKAGSDELTVKNGYLTGTIQGREVVVLYGDPTSQITFTVNNATTVWGQNVYVVGNISQLGNWDPVHAVQMTPSSYPTWTVTIPLLQGQNIQFKFIKKDSAGNVIWEDISNRTYTVPTAASGAYTASWNVPHHHHHH

SEQ ID NO: 28 - DNA, 1731 bp, Artificial Sequence
  Hybrid polypeptide comprising Lactobacillus amylovorus catalytic domain
  sig_peptide (1)..(81)
  misc_structure (82)..(1731)
atgaagaaaccgttggggaaaattgtcgcaagcaccgcactactcatttctgttgcttttagttcatcgatagcatcagcagctagtgatacgacatcaactgatcactcaagcaatgatacagctgattctgttagcgacggtgttattttgcatgcatggtgctggtcgttcaacacgattaaaaacaacttgaaacagattcatgacgccggctacacagcggttcaaacttcacctgttaatgaagttaaagttggaaatagcgggtctaagtcattaaataactggtattggctatatcagccaactaaatatagtattggtaactattatttaggaacggaagctgaatttaagtcaatgtgcgctgctgctaaagaatataatatcaggatcattgtcgatgcaactctgaatgatacaacaagtgattatagtgcaatttcggatgaaattaaaagtattccaaattggacacatggtaacaaacaaatttcgaattggagtgatcgtgaagatgttactcaaaattcgttgttaggtttatatgattggaatactcaaaattctcaagtccagacgtatttgaagaattatttggaacgcttgatttctgacggagcttcaggctttcgttatgatgcagctaagcatattgaacttccaagtcaatatgatggcagctatggcagcaatttctggccaaatattactgataatgggtctgaatttcagtatggtgaagttttgcaggactcgatttcaaaagaatcagattatgctaattacatgagtgttacagcttcaaattacggcaatacgattcgcaatgcgttaaagaatcgtgattttaccgcaagtactttgcagaatttcaacatcagtgttccagcttctaaattagtaacttgggtcgaatcgcatgataattatgctaacgatgatcaagtttcgacttggatgaataatagtgatattaaattaggctgggctgttgttgcttcgcgttctggtagtgttccgctgttctttgaccgtccagttgatggtggtaatggtactcggttccctggcagttcagaaattggtgatgctggcagcagtttgtattatgataaagcagttgtagctgttaataaattccataatgcaatggctggtcaatctgaatatatttctaatccaaatggcaataccaagatttttgaaaatgaacgtggcagcaaaggggttgtttttgcaaacgcttccgacagttcatatagtttgaatgttaaaactagtttagctgatgggacttatgaaaacaaggctggttcagatgaacttaccgttaaaaatggttatttaaccggtacaattcaaggacgtgaagttgttgttctttacggggatccaccggaaccgacgccggagccgcaaataacatttactgtaaataacgccacaaccgtttggggacaaaatgtatacgttgtcgggaatatttcgcagctggggaactgggatccagtccacgcagttcaaatgacgccgtcttcttatccaacatggactgtaacaatccctcttcttcaagggcaaaacatacaatttaaatttatcaaaaaagattcagctggaaatgtcatttgggaagatatatcgaatcgaacatacaccgtcccaactgctgcatccggagcatatacagccagctggaacgtgccccaccatcaccatcatcac

SEQ ID NO: 29 - protein, 577 aa, Artificial Sequence
  Hybrid polypeptide comprising Lactobacillus amylovorus catalytic domain
MKKPLGKIVASTALLISVAFSSSIASAASDTTSTDHSSNDTADSVSDGVILHAWCWSFNTIKNNLKQIHDAGYTAVQTSPVNEVKVGNSGSKSLNNWYWLYQPTKYSIGNYYLGTEAEFKSMCAAAKEYNIRIIVDATLNDTTSDYSAISDEIKSIPNWTHGNKQISNWSDREDVTQNSLLGLYDWNTQNSQVQTYLKNYLERLISDGASGFRYDAAKHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNNSDIKLGWAVVASRSGSVPLFFDRPVDGGNGTRFPGSSEIGDAGSSLYYDKAVVAVNKFHNAMAGQSEYISNPNGNTKIFENERGSKGVVFANASDSSYSLNVKTSLADGTYENKAGSDELTVKNGYLTGTIQGREVVVLYGDPPEPTPEPQITFTVNNATTVWGQNVYVVGNISQLGNWDPVHAVQMTPSSYPTWTVTIPLLQGQNIQFKFIKKDSAGNVIWEDISNRTYTVPTAASGAYTASWNVPHHHHHH

SEQ ID NO: 30 - protein, 550 aa, Artificial Sequence
  Hybrid polypeptide comprising Lactobacillus amylovorus catalytic domain
ASDTTSTDHSSNDTADSVSDGVILHAWCWSFNTIKNNLKQIHDAGYTAVQTSPVNEVKVGNSGSKSLNNWYWLYQPTKYSIGNYYLGTEAEFKSMCAAAKEYNIRIIVDATLNDTTSDYSAISDEIKSIPNWTHGNKQISNWSDREDVTQNSLLGLYDWNTQNSQVQTYLKNYLERLISDGASGFRYDAAKHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNNSDIKLGWAVVASRSGSVPLFFDRPVDGGNGTRFPGSSEIGDAGSSLYYDKAVVAVNKFHNAMAGQSEYISNPNGNTKIFENERGSKGVVFANASDSSYSLNVKTSLADGTYENKAGSDELTVKNGYLTGTIQGREVVVLYGDPPEPTPEPQITFTVNNATTVWGQNVYVVGNISQLGNWDPVHAVQMTPSSYPTWTVTIPLLQGQNIQFKFIKKDSAGNVIWEDISNRTYTVPTAASGAYTASWNVPHHHHHH

SEQ ID NO: 31 - DNA, 2271 bp, Artificial Sequence
  Hyrbid polypeptide comprising Lactobacillus amylovorus catalytic domain
  sig_peptide (1)..(81)
  misc_structure (82)..(2271): mature polypeptide coding sequence
atgaagaaaccgttggggaaaattgtcgcaagcaccgcactactcatttctgttgcttttagttcatcgatagcatcagcagctagtgatacgacatcaactgatcactcaagcaatgatacagctgattctgttagcgacggtgttattttgcatgcatggtgctggtcgttcaacacgattaaaaacaacttgaaacagattcatgacgccggctacacagcggttcaaacttcacctgttaatgaagttaaagttggaaatagcgggtctaagtcattaaataactggtattggctatatcagccaactaaatatagtattggtaactattatttaggaacggaagctgaatttaagtcaatgtgcgctgctgctaaagaatataatatcaggatcattgtcgatgcaactctgaatgatacaacaagtgattatagtgcaatttcggatgaaattaaaagtattccaaattggacacatggtaacaaacaaatttcgaattggagtgatcgtgaagatgttactcaaaattcgttgttaggtttatatgattggaatactcaaaattctcaagtccagacgtatttgaagaattatttggaacgcttgatttctgacggagcttcaggctttcgttatgatgcagctaagcatattgaacttccaagtcaatatgatggcagctatggcagcaatttctggccaaatattactgataatgggtctgaatttcagtatggtgaagttttgcaggactcgatttcaaaagaatcagattatgctaattacatgagtgttacagcttcaaattacggcaatacgattcgcaatgcgttaaagaatcgtgattttaccgcaagtactttgcagaatttcaacatcagtgttccagcttctaaattagtaacttgggtcgaatcgcatgataattatgctaacgatgatcaagtttcgacttggatgaataatagtgatattaaattaggctgggctgttgttgcttcgcgttctggtagtgttccgctgttctttgaccgtccagttgatggtggtaatggtactcggttccctggcagttcagaaattggtgatgctggcagcagtttgtattatgataaagcagttgtagctgttaataaattccataatgcaatggctggtcaatctgaatatatttctaatccaaatggcaataccaagatttttgaaaatgaacgtggcagcaaaggggttgtttttgcaaacgcttccgacagttcatatagtttgaatgttaaaactagtttagctgatgggacttatgaaaacaaggctggttcagatgaacttaccgttaaaaatggttatttaaccggtacaattcaaggacgtgaagttgttgttctttacggggatccaccggaaccgacgccggagccgcctcatgtctttcttgagaattaccaaacgggggcagtccattctttcaatgatcagctgacggtcaccctgcgtgcaaatgcgaaaacaacaaaagccgtttaccaaatcaataatgggcagcagacagcatttaaggatggagaccgactaacgatcgggaaaggagatccaatcggcacgacatacaacatcaaattaaccggaacgaacggcgagggtgcagcgaggacccaagaatacaagtttgttaaaaaagacccgaaaaaggtgtactttgaaaaaccaagcagttggggaagcagagtttacgcgtatgtctacaataagaacacaaataaagcaattacctcggcttggccgggcaaaaaaatgacggcgcttggaaatgataaatacgaattagatctggacaccgatgaagatgactccgatttagcggtcattttcaccgatgggacaaaacaaacgccggccgctaatgaagcaggcttcacattcacagcggatgcgacgtacgatcaaaatggcgttgtgaaacaaataacatttactgtaaataacgccacaaccgtttggggacaaaatgtatacgttgtcgggaatatttcgcagctggggaactgggatccagtccacgcagttcaaatgacgccgtcttcttatccaacatggactgtaacaatccctcttcttcaagggcaaaacatacaatttaaatttatcaaaaaagattcagctggaaatgtcatttgggaagatatatcgaatcgaacatacaccgtcccaactgctgcatccggagcatatacagccagctggaacgtgccccaccatcaccatcatcac

SEQ ID NO: 32 - protein, 757 aa, Artificial Sequence
  Hybrid polypeptide comprising Lactobacillus amylovorus catalytic domain
MKKPLGKIVASTALLISVAFSSSIASAASDTTSTDHSSNDTADSVSDGVILHAWCWSFNTIKNNLKQIHDAGYTAVQTSPVNEVKVGNSGSKSLNNWYWLYQPTKYSIGNYYLGTEAEFKSMCAAAKEYNIRIIVDATLNDTTSDYSAISDEIKSIPNWTHGNKQISNWSDREDVTQNSLLGLYDWNTQNSQVQTYLKNYLERLISDGASGFRYDAAKHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNNSDIKLGWAVVASRSGSVPLFFDRPVDGGNGTRFPGSSEIGDAGSSLYYDKAVVAVNKFHNAMAGQSEYISNPNGNTKIFENERGSKGVVFANASDSSYSLNVKTSLADGTYENKAGSDELTVKNGYLTGTIQGREVVVLYGDPPEPTPEPPHVFLENYQTGAVHSFNDQLTVTLRANAKTTKAVYQINNGQQTAFKDGDRLTIGKGDPIGTTYNIKLTGTNGEGAARTQEYKFVKKDPKKVYFEKPSSWGSRVYAYVYNKNTNKAITSAWPGKKMTALGNDKYELDLDTDEDDSDLAVIFTDGTKQTPAANEAGFTFTADATYDQNGVVKQITFTVNNATTVWGQNVYVVGNISQLGNWDPVHAVQMTPSSYPTWTVTIPLLQGQNIQFKFIKKDSAGNVIWEDISNRTYTVPTAASGAYTASWNVPHHHHHH

SEQ ID NO: 33 - protein, 730 aa, Artificial Sequence
  Hybrid polypeptide comprising Lactobacillus amylovorus catalytic domain
ASDTTSTDHSSNDTADSVSDGVILHAWCWSFNTIKNNLKQIHDAGYTAVQTSPVNEVKVGNSGSKSLNNWYWLYQPTKYSIGNYYLGTEAEFKSMCAAAKEYNIRIIVDATLNDTTSDYSAISDEIKSIPNWTHGNKQISNWSDREDVTQNSLLGLYDWNTQNSQVQTYLKNYLERLISDGASGFRYDAAKHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNNSDIKLGWAVVASRSGSVPLFFDRPVDGGNGTRFPGSSEIGDAGSSLYYDKAVVAVNKFHNAMAGQSEYISNPNGNTKIFENERGSKGVVFANASDSSYSLNVKTSLADGTYENKAGSDELTVKNGYLTGTIQGREVVVLYGDPPEPTPEPPHVFLENYQTGAVHSFNDQLTVTLRANAKTTKAVYQINNGQQTAFKDGDRLTIGKGDPIGTTYNIKLTGTNGEGAARTQEYKFVKKDPKKVYFEKPSSWGSRVYAYVYNKNTNKAITSAWPGKKMTALGNDKYELDLDTDEDDSDLAVIFTDGTKQTPAANEAGFTFTADATYDQNGVVKQITFTVNNATTVWGQNVYVVGNISQLGNWDPVHAVQMTPSSYPTWTVTIPLLQGQNIQFKFIKKDSAGNVIWEDISNRTYTVPTAASGAYTASWNVPHHHHHH

SEQ ID NO: 34 - DNA, 1728 bp, Artificial Sequence
  polypeptide having alpha-amylase activity comprising catalytic domain from Lactobacillus amylovorus
atgaaaaaaccgctggggaaaattgtcgcaagcaccgcactactcatttctgttgcttttagttcatcgatcgcatcggctgctagtgatacgacatcaactgatcactcaagcaatgatacagctgattctgttagcgacggtgttattttgcatgcatggtgctggtcgttcaacacgattaaaaacaacttgaaacagattcatgacgccggctacacagcggttcaaacttcacctgttaatgaagttaaagttggaaatagcgggtctaagtcattaaataactggtattggctatatcagccaactaaatatagtattggtaactattatttaggaacggaagctgaatttaagtcaatgtgcgctgctgctaaagaatataatatcaggatcattgtcgatgcaactctgaatgatacaacaagtgattatagtgcaatttcggatgaaattaaaagtattccaaattggacacatggtaacaaacaaatttcgaattggagtgatcgtgaagatgttactcaaaattcgttgttaggtttatatgattggaatactcaaaattctcaagtccagacgtatttgaagaattatttggaacgcttgatttctgacggagcttcaggctttcgttatgatgcagctaagcatattgaacttccaagtcaatatgatggcagctatggcagcaatttctggccaaatattactgataatgggtctgaatttcagtatggtgaagttttgcaggactcgatttcaaaagaatcagattatgctaattacatgagtgttacagcttcaaattacggcaatacgattcgcaatgcgttaaagaatcgtgattttaccgcaagtactttgcagaatttcaacatcagtgttccagcttctaaattagtaacttgggtcgaatcgcatgataattatgctaacgatgatcaagtttcgacttggatgaataatagtgatattaaattaggctgggctgttgttgcttcgcgttctggtagtgttccgctgttctttgaccgtccagttgatggtggtaatggtactcggttccctggcagttcagaaattggtgatgctggcagcagtttgtattatgataaagcagttgtagctgttaataaattccataatgcaatggctggtcaatctgaatatatttctaatccaaatggcaataccaagatttttgaaaatgaacgtggcagcaaaggggttgtttttgcaaacgcttccgacagttcatatagtttgaatgttaaaactagtttagctgatgggacttatgaaaacaaggctggttcagatgaacttaccgttaaaaatggttatttaaccggtacaattcaaggacgtgaagttgttgttctttacggggatccatccggcaactctggtggcccaactgtagcagtaacattcaacgttaacgcagagactgtatggggtgagaacatctaccttacgggcagcgttgacgcacttgagaactggtcagcagacaacgctcttcttctttcttcagctaactacccaacgtggtctatcacggttaaccttcctgctagcacggctatcgagtacaaatacatccgcaaaaacaatggcgcagttacatgggagtctgacccaaacaactctatcactacaccagcttcaggctctacaacagagaacgacacatggcgccatcatcaccatcaccactaa

SEQ ID NO: 35 - protein, 575 aa, Artificial Sequence
  polypeptide having alpha-amylase activity comprising catalytic domain from Lactobacillus amylovorus.
MKKPLGKIVASTALLISVAFSSSIASAASDTTSTDHSSNDTADSVSDGVILHAWCWSFNTIKNNLKQIHDAGYTAVQTSPVNEVKVGNSGSKSLNNWYWLYQPTKYSIGNYYLGTEAEFKSMCAAAKEYNIRIIVDATLNDTTSDYSAISDEIKSIPNWTHGNKQISNWSDREDVTQNSLLGLYDWNTQNSQVQTYLKNYLERLISDGASGFRYDAAKHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNNSDIKLGWAVVASRSGSVPLFFDRPVDGGNGTRFPGSSEIGDAGSSLYYDKAVVAVNKFHNAMAGQSEYISNPNGNTKIFENERGSKGVVFANASDSSYSLNVKTSLADGTYENKAGSDELTVKNGYLTGTIQGREVVVLYGDPSGNSGGPTVAVTFNVNAETVWGENIYLTGSVDALENWSADNALLLSSANYPTWSITVNLPASTAIEYKYIRKNNGAVTWESDPNNSITTPASGSTTENDTWRHHHHHH

SEQ ID NO: 36 - protein, 548 aa, Artificial Sequence
  polypeptide having alpha-amylase activity comprising catalytic domain from Lactobacillus amylovorus
ASDTTSTDHSSNDTADSVSDGVILHAWCWSFNTIKNNLKQIHDAGYTAVQTSPVNEVKVGNSGSKSLNNWYWLYQPTKYSIGNYYLGTEAEFKSMCAAAKEYNIRIIVDATLNDTTSDYSAISDEIKSIPNWTHGNKQISNWSDREDVTQNSLLGLYDWNTQNSQVQTYLKNYLERLISDGASGFRYDAAKHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNNSDIKLGWAVVASRSGSVPLFFDRPVDGGNGTRFPGSSEIGDAGSSLYYDKAVVAVNKFHNAMAGQSEYISNPNGNTKIFENERGSKGVVFANASDSSYSLNVKTSLADGTYENKAGSDELTVKNGYLTGTIQGREVVVLYGDPSGNSGGPTVAVTFNVNAETVWGENIYLTGSVDALENWSADNALLLSSANYPTWSITVNLPASTAIEYKYIRKNNGAVTWESDPNNSITTPASGSTTENDTWRHHHHHH

SEQ ID NO: 37 - DNA, 1737 bp, Artificial Sequence
  encoding polypeptide having alpha-amylase activity comprising catalytic domain from Lactobacillus amylovorus
atgaaaaaaccgctggggaaaattgtcgcaagcaccgcactactcatttctgttgcttttagttcatcgatcgcatcggctgctagtgatacgacatcaactgatcactcaagcaatgatacagctgattctgttagcgacggtgttattttgcatgcatggtgctggtcgttcaacacgattaaaaacaacttgaaacagattcatgacgccggctacacagcggttcaaacttcacctgttaatgaagttaaagttggaaatagcgggtctaagtcattaaataactggtattggctatatcagccaactaaatatagtattggtaactattatttaggaacggaagctgaatttaagtcaatgtgcgctgctgctaaagaatataatatcaggatcattgtcgatgcaactctgaatgatacaacaagtgattatagtgcaatttcggatgaaattaaaagtattccaaattggacacatggtaacaaacaaatttcgaattggagtgatcgtgaagatgttactcaaaattcgttgttaggtttatatgattggaatactcaaaattctcaagtccagacgtatttgaagaattatttggaacgcttgatttctgacggagcttcaggctttcgttatgatgcagctaagcatattgaacttccaagtcaatatgatggcagctatggcagcaatttctggccaaatattactgataatgggtctgaatttcagtatggtgaagttttgcaggactcgatttcaaaagaatcagattatgctaattacatgagtgttacagcttcaaattacggcaatacgattcgcaatgcgttaaagaatcgtgattttaccgcaagtactttgcagaatttcaacatcagtgttccagcttctaaattagtaacttgggtcgaatcgcatgataattatgctaacgatgatcaagtttcgacttggatgaataatagtgatattaaattaggctgggctgttgttgcttcgcgttctggtagtgttccgctgttctttgaccgtccagttgatggtggtaatggtactcggttccctggcagttcagaaattggtgatgctggcagcagtttgtattatgataaagcagttgtagctgttaataaattccataatgcaatggctggtcaatctgaatatatttctaatccaaatggcaataccaagatttttgaaaatgaacgtggcagcaaaggggttgtttttgcaaacgcttccgacagttcatatagtttgaatgttaaaactagtttagctgatgggacttatgaaaacaaggctggttcagatgaacttaccgttaaaaatggttatttaaccggtacaattcaaggacgtgaagttgttgttctttacggggatccatccagcaacagttgcggaggcggaggatcgactgtcgccgtgacgttcaacgtgaacgcccaaacggtttggggcgaaaacatctacatcactggctcggttgacgctctgagtaactggtctcccgacaacgccctcttgctctcgtctgccaactacccgacctggagcattaccgtgaatttacccgcgagcactgccattcagtataagtatatccgcaagatgaacggagctgtcacctgggaatccgatcccaacaacagcataactactccagccagcggctcctggaccgagaatgacacttggcgtcatcatcaccatcaccactaa

SEQ ID NO: 38 - protein, 578 aa, Artificial Sequence
  hybrid polypeptide having alpha-amylase activity comprising catalytic domain from Lactobacillus amylovorus
MKKPLGKIVASTALLISVAFSSSIASAASDTTSTDHSSNDTADSVSDGVILHAWCWSFNTIKNNLKQIHDAGYTAVQTSPVNEVKVGNSGSKSLNNWYWLYQPTKYSIGNYYLGTEAEFKSMCAAAKEYNIRIIVDATLNDTTSDYSAISDEIKSIPNWTHGNKQISNWSDREDVTQNSLLGLYDWNTQNSQVQTYLKNYLERLISDGASGFRYDAAKHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNNSDIKLGWAVVASRSGSVPLFFDRPVDGGNGTRFPGSSEIGDAGSSLYYDKAVVAVNKFHNAMAGQSEYISNPNGNTKIFENERGSKGVVFANASDSSYSLNVKTSLADGTYENKAGSDELTVKNGYLTGTIQGREVVVLYGDPSSNSCGGGGSTVAVTFNVNAQTVWGENIYITGSVDALSNWSPDNALLLSSANYPTWSITVNLPASTAIQYKYIRKMNGAVTWESDPNNSITTPASGSWTENDTWRHHHHHH

SEQ ID NO: 39 - protein, 551 aa, Artificial Sequence
  hybrid polypeptide having alpha-amylase activity comprising catalytic domain from Lactobacillus amylovorus
ASDTTSTDHSSNDTADSVSDGVILHAWCWSFNTIKNNLKQIHDAGYTAVQTSPVNEVKVGNSGSKSLNNWYWLYQPTKYSIGNYYLGTEAEFKSMCAAAKEYNIRIIVDATLNDTTSDYSAISDEIKSIPNWTHGNKQISNWSDREDVTQNSLLGLYDWNTQNSQVQTYLKNYLERLISDGASGFRYDAAKHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNNSDIKLGWAVVASRSGSVPLFFDRPVDGGNGTRFPGSSEIGDAGSSLYYDKAVVAVNKFHNAMAGQSEYISNPNGNTKIFENERGSKGVVFANASDSSYSLNVKTSLADGTYENKAGSDELTVKNGYLTGTIQGREVVVLYGDPSSNSCGGGGSTVAVTFNVNAQTVWGENIYITGSVDALSNWSPDNALLLSSANYPTWSITVNLPASTAIQYKYIRKMNGAVTWESDPNNSITTPASGSWTENDTWRHHHHHH